(19) United States
(12) Patent Application Publication
(10) Pub. No.: US 2011/0066175 A1
(43) Pub. Date: Mar. 17, 2011

(54) GASTRIC ANCHOR

(75) Inventor: Yossi GROSS, Moshav Mazor (IL)

(73) Assignee: RAINBOW MEDICAL LTD., Herzliya (IL)

(21) Appl. No.: 12/940,790

(22) Filed: Nov. 5, 2010

Related U.S. Application Data
(63) Continuation-in-part of application No. 12/793,978, filed on Jun. 4, 2010.
Application No. 12/793,978 is a continuation-in-part of application No. PCT/IL2010/000230, filed on Mar. 21, 2010.
Application No. PCT/IL2010/000230 is a continuation-in-part of application No. 12/437,250, filed on May 7, 2009.

(51) Int. Cl. A61M 29/00

(52) U.S. Cl. 606/191

(57) ABSTRACT

A swallowable medical treatment device is configured to initially assume a contracted state having a volume of less than 4 cm3. The device includes a gastric anchor, which initially assumes a contracted size, and which is configured to, upon coming in contact with a liquid, expand sufficiently to prevent passage of the gastric anchor through a round opening having a diameter of between 1 cm and 3 cm. The device also includes a duodenal unit, which is configured to pass through the opening, and which is coupled to the gastric anchor such that the duodenal unit is held between 1 cm and 20 cm from the gastric anchor. Other embodiments are also described.

### CROSS-REFERENCE TO RELATED APPLICATIONS

The present application claims priority from and is a continuation-in-part of U.S. application Ser. No. 12/793,978, filed Jun. 4, 2010, which is a continuation-in-part of International Patent Application PCT/IL2010/000230, filed Mar. 21, 2010, which is a continuation-in-part of U.S. application Ser. No. 12/437,250, filed May 7, 2009, all of which are assigned to the assignee of the present application and are incorporated herein by reference.

### FIELD OF THE APPLICATION

The present invention relates generally to medical devices, and specifically to gastroretentive devices.

### BACKGROUND OF THE APPLICATION

Gastroretentive dosage forms (GRDFs) are swallowable drug delivery dosage forms having a prolonged gastric residence time, which substantially increases the time period during which the drug is released. Expandable GRDFs assume an initial, swallowable size, and expand in the stomach to a larger size that delays passage from the stomach.

Klausner E A et al., in “Expandable gastroretentive dosage forms,” Journal of Controlled Release 90:143-162 (2003), which is incorporated herein by reference, survey expandable GRDFs as reported in articles and patents.

U.S. Pat. No. 6,776,999 to Krumme describes a device for delaying the pylorus passage of orally administered medicament forms. The device comprises a component which expands upon contact with the gastric juice and a polymer coat which is permeable to liquids but not to gases. The device can contain an active substance whose release into the gastric juice is mainly controlled by the medicament form into which it is incorporated. The device can be easily rolled or folded and can be filled into capsules.

U.S. Pat. No. 4,878,905 to Blass describes a nonsurgically implanted gastrointestinal module. The module consists of an ellipsoidal or spherical collapsible gastric anchor, a tether device, and an intestinal payload module. The device is inserted into the stomach via a gastric intubation technique. The gastric anchor unfolds within the gastric cavity and lodges itself prior to the sphincter. The smaller intestinal module passes through the sphincter and unfolds within the confines of the intestine. The intestinal module is held in place via a tether which binds the gastric anchor to the intestinal payload module. The intestinal module may contain slow release medicaments, bound enzymes, cofactors, buffers, microorganisms and the like. Thus biochemical processes of the intestine may be modified, and these in turn may affect other body compartments. The gastrointestinal module may be removed with a retrieval hook via a gastric intubation technique.

U.S. Pat. No. 4,767,627 to Caldwell et al. describes a drug delivery device retained in the stomach comprising a planar figure made from an erodible polymer that may release a drug associated therewith over a controlled, predictable and extended period of time.

U.S. Pat. No. 6,685,962 to Friedman et al. describes pharmaceutical gastroretentive drug delivery systems for the controlled release of an active agent in the gastrointestinal tract, which comprise: (a) a single- or multi-layered matrix comprising a polymer that does not retain in the stomach more than a conventional dosage form selected from (1) degradable polymers that may be hydrophilic polymers not instantly soluble in gastric fluids, enteric polymers substantially insoluble at pH less than 5.5 and/or hydrophobic polymers and mixtures thereof; (2) non-degradable polymers; and any mixtures of (1) and (2); (b) a continuous or non-continuous membrane comprising at least one polymer having a substantial mechanical strength; and (c) a drug; wherein the matrix when affixed or attached to the membrane prevents evacuation from the stomach of the delivery system for a period of time of between about 3 to about 24 hours.

US Patent Application Publication 2004/0180086 to Ramtoola et al. describes gastro-retentive dosage forms for prolonged delivery of levodopa and carbidopalevodopa combinations. The dosage forms comprise a tablet containing the active ingredient and a gas-generating agent sealed within an expandable, hydrophilic, water-permeable and substantially gas-impermeable membrane. Upon contact with gastric fluid, the membrane expands as a result of the release of gas from the gas-generating agent in the tablet. The expanded membrane is retained in the stomach for a prolonged period of time up to 24 hours or more during which period the active ingredient is released from the tablet providing delivery of levodopa to the site of optimum absorption in the upper small intestine.

U.S. Pat. No. 6,994,095 to Burnett describes pyloric valve corking devices and methods. The devices generally include an occluding member which expands from a first configuration to a larger second configuration and a bridging member extending from the occluding member. The bridging member has a length which passes at least partially through the gastric opening such that the occluding member obstructs the gastric opening, and wherein the length permits the occluding member to intermittently move relative to the gastric opening. A second occluding member may be attached to the distal end of the bridging member. The reduction in flow of gastric contents into the duodenum can be tightly regulated using a pump or valve. Otherwise, the flow can be passively regulated with the occluding device.

PCT Publication WO 2008/121409 to Vargas describes an intragastric implant comprising an anchor and a therapeutic device or a diagnostic device. The anchor is adapted to extend between the fundus and the pyloric valve of a stomach, to be retained without attachment to the stomach wall, and to anchor the device within the stomach with a relatively stable position and orientation. The therapeutic or diagnostic device is adapted to extend from the esophagus or stomach to the intestines or stomach. The therapeutic or diagnostic device, when extending into the esophagus, is slidably received through the gastroesophageal junction and, when extending into the intestines, is slidably received in the pyloric valve.

US Patent Application Publication 2007/0293885 to Binmoeller describes an intestinal/duodenal insert comprising an elongated member with at least one flow reduction element that can cause the stimulation of one or more biological signals of satiety. Some embodiments of the inserted device are anchored at the duodenal site by an anchoring member residing in the stomach, while other embodiments of the device are stabilized at a targeted site by appropriate dimensions of length as well as one or more angled portions of the device that correspond to angled portions of the targeted site in the duodenum. Embodiments of the device exert effects by virtue of physical presence, as well as by more active forms of intervention, including release of bioactive materials and electrical stimulation of neurons.

PCT Publication WO 2008/154450 to Swain et al. describes techniques for attaching or maintaining the position of a therapeutic or diagnostic device in a body lumen, such as the GI tract, without necessarily requiring any penetrating attachments through any body walls. The system includes at least two elements: a proximal orientation element and a distal support element.

Gastric space fillers are known for filling a portion of the stomach, thereby reducing available space for food, and creating a feeling of satiety.

US Patent Application Publication 2007/0156248 to Marco et al. describes bioerodible, biodegradable, or digestible self-deploying intragastric implants that may be swallowed. Once swallowed, the implants undergo self-expansion in the stomach and apply a suitable pressure against the stomach wall to provide a feeling of satiety to the individual. The implants then dissolve or are disassembled perhaps using gastric liquids and pass out of the stomach.

PCT Publication WO 2008/121831 to Quijana et al. describes gastric space filler device for treating obesity in a patient by reducing the stomach volume features at least one inflatable space filler with drug delivery and stimulation features and includes therapeutic devices and anchoring apparatus enabling tracking, visualization and optimized management of inter-balloon connecting sections, drug reservoirs and pumping systems.

US Patent Application Publication 2006/0142731 to Brooks describes a floating anchor, which can be inserted into the esophagus, stomach, small intestine, large intestine, or rectal cavity and reverts to a bent shape when placed therein.

PCT Publication WO 2008/023374 to Shalon et al. describes a device for modifying an eating behavior of a subject. The device includes a device body which is attachable to GI tract tissue of a subject and functions in altering an eating behavior thereof.

PCT Publication WO 2007/007339 to Gross et al. describes a method including placing first and second electrodes at respective first and second sites of a duodenum of a subject, and activating the electrodes to increase a blood insulin level of the subject or to induce or increase a rate of peristalsis in the duodenum.

Sun et al., in “Intestinal electric stimulation decreases fat absorption in rats: Therapeutic potential for obesity,” Obes Res. 2004 August; 12(8):1235-42, which is incorporated herein by reference, describe a study investigating whether intestinal electric stimulation (IES) would reduce fat absorption and, thus, would be a potential therapy for obesity.

U.S. Pat. No. 7,267,694 to Levine et al. describes techniques for limiting absorption of food products in specific parts of the digestive system. A gastrointestinal implant device is anchored in the stomach and extends beyond the ligament of Treitz. All food exiting the stomach is funneled through the device. The gastrointestinal device includes an anchor for anchoring the device to the stomach and a flexible sleeve to limit absorption of nutrients in the duodenum. The anchor is collapsible for endoscopic delivery and removal.

PCT Publication WO 06/064503 to Belsky et al. describes apparatus for drug administration, including an ingestible capsule, which includes a drug, stored by the capsule. The apparatus also includes an environmentally-sensitive mechanism, adapted to change a state thereof responsively to a disposition of the capsule within a gastrointestinal (GI) tract of a subject; one or more drug-passage facilitation electrodes; and a control component, adapted to facilitate passage of the drug, in response to a change of state of the environmentally-sensitive mechanism, by driving the drug-passage facilitation electrodes to apply an electrical current. The apparatus further includes a velocity-reduction element adapted to reduce a velocity of the capsule through the GI tract for at least a portion of the time that the control component is facilitating the passage of the drug.

PCT Publication WO/1994/001165 to Gross describes a medication administering device that includes a housing introducible into a body cavity and of a material insoluble in the body cavity fluids, but formed with an opening covered by a material which is soluble in body cavity fluids. A diaphragm divides the interior of the housing into a medication chamber including the opening, and a control chamber. An electrolytic cell in the control chamber generates a gas when electrical current is passed therethrough to deliver medication from the medication chamber through the opening into the body cavity at a rate controlled by the electrical current. The device can be in the form of a pill or capsule to be taken orally.

The following references may be of interest:

U.S. Pat. No. 4,738,667 to Galloway

U.S. Pat. No. 5,188,104 to Wernicke et al.

U.S. Pat. No. 5,536,274 to Neuss

U.S. Pat. No. 6,183,461 to Matsuura et al.

U.S. Pat. No. 6,364,868 to Ikeguchi

U.S. Pat. No. 6,743,198 to Tihon

U.S. Pat. No. 7,320,696 to Gazi et al.

U.S. Pat. No. 7,476,256 to Meade et al.

US Patent Application Publication 2003/0021845 to Friedman et al.

US Patent Application Publication 2003/0191492 to Gellman

US Patent Application Publication 2005/0096750 to Kagan et al.

US Patent Application Publication 2007/0005147 to Levine et al.

US Patent Application Publication 2007/0250132 to Burnett

US Patent Application Publication 2008/0063703 to Gross et al.

US Patent Application Publication 2008/0234834 to Meade et al.

US Patent Application Publication 2008/0281375 to Chen

### SUMMARY OF APPLICATIONS

In some embodiments of the present invention, a swallowable treatment device comprises a gastric anchor coupled to a duodenal unit configured to reside in the duodenum. The treatment device, including the gastric anchor, initially assumes a contracted swallowable configuration. After the device is swallowed and enters the stomach, the anchor expands to prevent passage of the anchor through the pylorus even when the pylorus is in an open, relaxed state. The duodenal unit passes into the duodenum and is prevented by the gastric anchor from passing further into the gastrointestinal (GI) tract. At least a portion of the anchor eventually biodegrades in the stomach, causing the anchor to break down, and the entire device to be evacuated through the GI tract by peristalsis. For some applications, the treatment device further comprises a tether which couples the duodenal unit to the gastric anchor.

For some applications, the duodenal unit is configured to dispense a drug. For some applications, the duodenal unit comprises a conventional drug pill comprising the drug. The pill may comprise, for example, a capsule. Alternatively, the duodenal unit may comprise a slow-release reservoir that slowly releases the drug into the duodenum. Alternatively or additionally, for some applications, the gastric anchor may be configured to dispense a drug. For some applications, the gastric anchor comprises a conventional drug pill comprising the drug. The pill may comprise, for example, a capsule. Alternatively, the gastric anchor may comprise a slow-release reservoir that slowly releases the drug into the stomach.

For some applications, the duodenal unit comprises two or more duodenal stimulation electrodes that are configured to come in physical contact with the wall of at least a portion of the duodenum. The treatment device comprises a power source, such as a battery, and circuitry that is configured to drive the electrodes to apply an electrical current to the wall of the duodenum, and to configure the current to induce and/or increase a rate of peristalsis in the duodenum, and/or induce migrating motor complex (MMC) in the duodenum. As a result, the residence time of absorbable food calories in the duodenum is reduced. For some applications, the duodenal unit is shaped so as to define a passage therethrough, through which chyme can pass.

For some applications, the duodenal unit comprises a bariatric sleeve sized to allow chyme to pass therethrough without coming into contact with the wall of at least a portion of the duodenum. Such bypassing of the duodenum reduces absorption of nutrients and calories. Optionally, the sleeve is long enough to additionally bypass a portion of the jejunum. The sleeve is typically biodegradable, such that after a period of time the sleeve degrades and is evacuated through the GI tract by peristalsis. The sleeve is typically coupled directly to the anchor, so that in these applications the tether is typically not provided. For some applications, the duodenal unit is initially shaped to have a rounded tip, which facilitates passage through the pylorus.

For some applications, the gastric anchor is configured to serve as a pyloric plug, which is configured to at least partially block the pylorus. After the treatment device is swallowed, peristalsis advances the device toward the pylorus, and the duodenal unit into the duodenum. Peristalsis in the duodenum advances the duodenal unit in the duodenum, causing the duodenal unit to pull the pyloric plug toward the pylorus, until the pyloric plug at least partially (e.g., fully) blocks the pylorus. The duodenal unit is prevented by the gastric anchor from passing further into the GI tract.

Such a partial or full blockage of the pylorus induces a sensation of satiety, e.g., by slowing the passage of chyme from the stomach. The device thus may slow a rise in blood sugar during and after eating food. The device is thus useful for treating conditions such as obesity and diabetes.

Typically, use of the pyloric plug results in intermittent, alternating periods of peristalsis in the duodenum, and corresponding periods of full or partial blocking of the pylorus. During periods in which chyme is not in the duodenum, and thus peristalsis does not occur (or occurs at a lesser rate or strength), the duodenal unit does not hold the pyloric plug against the pylorus. Natural muscular activity of the stomach moves the pyloric plug away from the pylorus, allowing chyme to pass through the pylorus. Passage of chyme through the pylorus into the duodenum causes duodenal peristalsis, which causes the duodenal unit to pull the pyloric plug against the pylorus, and/or against the wall of the antrum surrounding the pylorus. This opened/closed cycling of the pylorus reduces the rate of release of the chyme from the stomach into the duodenum.

For some applications, the duodenal unit comprises one or more elongated members, each of which typically has a length of between 1 and 20 cm. For example, the elongated members may comprise strings, springs, tubes, ribbons, or a combination of such elements. For some applications, duodenal peristalsis pulls the elongated members distally in the duodenum, causing the members to pull the pyloric plug against the pylorus, and/or against the wall of the antrum surrounding the pylorus. In some configurations, the elongated members are configured to expand upon coming in contact with a liquid in the duodenum. Such expansion may inhibit motion of chyme into and/or through the duodenum, which may slow gastric emptying and/or inhibit absorption of nutrients.

For some applications, the gastric anchor comprises a pyloric plug, which, when in an expanded state, is bowl-shaped, i.e., is concave with an opening on one side. For some applications, the pyloric plug comprises a frame to which a flexible sheet is coupled. The frame comprises a plurality of ribs.

For some applications, the pyloric plug is configured to partially block the pylorus. For example, the pyloric plug may be shaped so as to define a passage therethrough, through which chyme can pass to the pylorus and the duodenum. The passage is smaller than the orifice of the pylorus, thereby allowing some of the chyme to pass through the pylorus during peristalsis, but at a slower rate than would occur in the absence of the plug.

For some applications, the pyloric plug is configured to define a passage therethrough, which has a size that varies while the anchor is in its expanded state. For some applications, the size of the passage decreases in response to greater pulling by the duodenal unit. Thus, when chyme is present in the duodenum, duodenal peristalsis pulls on the duodenal unit, causing a reduction in the size of the passage, and a reduction of the amount of chyme that passes through the pylorus. As chyme passes out of the duodenum, peristalsis in the duodenum decreases, the duodenal unit pulls less on the pyloric plug, and the size of the passage increases. The device thus regulates the passage of chyme from the stomach into the duodenum.

For example, at least a portion of the pyloric plug may comprise a curved strip of elastic material shaped as a conical helix when the pyloric plug is in its resting state (i.e., when the duodenal unit is not pulling on the plug). The tether passes through at least a portion of the helix (typically, including a base of the helix) and couples the duodenal unit to a vertex of the conical helix. When the duodenal unit pulls the tether, the tether pulls the vertex toward the base of the helix, thereby at least partially collapsing the helix, and reducing a size of the passage therethrough. Such pulling sometimes entirely closes the passage.

At least a portion of the device eventually biodegrades in the stomach, causing the pyloric plug, duodenal unit, and/or tether to eventually break down, and the entire device to be evacuated through the GI tract by peristalsis.

For some applications, a variety of treatment devices are provided, calibrated based on time of disintegration and/or size of the passage through the pyloric plug. The physician selects the most appropriate calibration, based on the individual patient's condition and/or pyloric orifice size.

For some applications, the gastric anchor comprises a flexible sheet sized to prevent passage of the anchor through the pylorus. For some applications, the sheet is shaped so as to define a passage therethrough, through which chyme can pass to the pylorus and the duodenum. For other applications in which the anchor comprises a pyloric plug, the sheet does not define a passage therethrough, or defines a passage that is smaller than the pylorus when the pylorus is in its open, relaxed state. Before the device is swallowed, the sheet is rolled to assume a contracted swallowable configuration. Upon arriving in the stomach, the sheet unrolls and becomes positioned in the antrum of the stomach by gastric peristalsis. For some applications, the sheet is initially rolled around at least a portion of the duodenal unit.

For some applications, the treatment device implements two or more of these techniques. For example, the unit may comprise both the drug and the duodenal stimulation electrodes. The gastric anchor may or may not comprise the pyloric plug in combination with the other techniques described herein, such as drug release and/or duodenal stimulation.

There is therefore provided, in accordance with an application of the present invention, apparatus including a swallowable medical treatment device, which is configured to initially assume a contracted state having a volume of less than 4 cm3, and which includes:

a gastric anchor, which initially assumes a contracted size, and which is configured to, upon coming in contact with a liquid, expand sufficiently to prevent passage of the gastric anchor through a round opening having a diameter of between 1 cm and 3 cm; and

a duodenal unit, which is configured to pass through the opening, and which is coupled to the gastric anchor such that the duodenal unit is held between 1 cm and 20 cm from the gastric anchor.

For some applications, the duodenal unit is coupled to the gastric anchor such that the duodenal unit is held between 2 cm and 5 cm from the gastric anchor.

For some applications, the apparatus further includes a tether, which couples the duodenal unit to the gastric anchor, and has a length of between 1 cm and 20 cm.

For some applications, the duodenal unit includes one or more elongated members, each of which has a length of between 1 and 20 cm. For some applications, the elongated members are configured to expand upon coming in contact with a duodenal liquid. For example, the elongated members may include sponges and/or balloons.

For some applications, the duodenal unit has a volume of between 0.2 cc and 10 cc.

For some applications, the duodenal unit is configured to dispense a drug.

For some applications, the duodenal unit includes two or more duodenal stimulation electrodes that are configured to come in physical contact with a wall of the duodenum, and the treatment device further includes a power source and circuitry that is configured to drive the electrodes to apply an electrical current to the wall of the duodenum.

For some applications, the duodenal unit includes a bariatric sleeve sized to allow chyme to pass therethrough without coming into contact with a wall of at least a portion of the duodenum.

For some applications, the gastric anchor includes a flexible sheet which initially is rolled around at least a portion of the duodenal unit to assume the contracted size, and which is configured to prevent the passage of the anchor through the opening by unrolling upon coming in contact with the liquid.

For some applications, the gastric anchor includes a flexible sheet which initially is rolled to assume the contracted size, and which is configured to prevent passage of the anchor through the opening by unrolling upon coming in contact with the liquid, which flexible sheet is shaped so as to define a passage therethrough. For some applications, the passage is shaped as a hole having a radius of at least 0.4 cm.

For some applications, the gastric anchor is configured to serve as a plug, which is configured to at least partially block the opening. For some applications, the gastric anchor includes a duodenal plug component, which is configured to at least partially pass through the opening, and expand upon coming in contact with a duodenal liquid, so as to at least partially block the opening. For some applications, the duodenal unit is coupled to the gastric anchor via the duodenal plug component. For some applications, the duodenal unit includes one or more elongated members, which are coupled to the duodenal plug component.

For some applications, the duodenal unit includes one or more elongated members, each of which has a length of between 1 and 20 cm, and which are configured to expand upon coming in contact with a duodenal liquid. For some applications, the gastric anchor includes a flexible sheet, which may, for example, have an area of less than 30 cm2.

For some applications, the gastric anchor, when expanded, is bowl-shaped. For some applications, the gastric anchor includes a frame, which includes a plurality of ribs, to which the flexible sheet is coupled. For some applications, the flexible sheet has an area of less than 30 cm2 and/or an area of at least 3 cm2. For some applications, the bowl-shaped gastric anchor is shaped so as to define a rim, and the gastric anchor further includes a band, which is coupled to the rim, and configured to prevent inversion of the bowl-shaped gastric anchor. For some applications, the band is biodegradable, such that, upon degrading, the band no longer prevents the inversion of the bowl-shaped gastric anchor. For some applications, the bowl-shaped gastric anchor is shaped so as to define a rim having a perimeter of between 3 cm and 12 cm.

For some applications, the gastric anchor is configured to fully block the opening.

For some applications, the device is configured to intermittently at least partially block the opening.

For some applications, the gastric anchor is configured to define a passage therethrough, which has a size that varies after the anchor has expanded. For some applications, the gastric anchor is configured such that a size of the passage decreases in response to pulling on the gastric anchor by the duodenal unit. For some applications, at least a portion of the gastric anchor is shaped so as to define a conical helix when in a resting state. For some applications, the apparatus further includes a tether, which passes through at least a portion of the conical helix, and couples the duodenal unit to a vertex of the conical helix.

For some applications, the gastric anchor, when expanded, is shaped as a sphere.

For some applications, the apparatus further includes a dissolvable enclosure that entirely surrounds the swallowable medical treatment device when the device initially assumes the contracted state.

For some applications, the opening is a pylorus of a subject, the liquid is stomach contents of the subject, the gastric anchor is configured to, upon coming in contact with the stomach contents, expand sufficiently to prevent passage of the anchor through the pylorus, and the duodenal unit is configured to pass through the pylorus, and is coupled to the gastric anchor such that the duodenal unit is held in a duodenum of the subject. For some applications, the gastric anchor is configured to at least partially biodegrade in a stomach of a subject, so as to allow passage of the anchor through the pylorus after a period of time.

There is further provided, in accordance with an application of the present invention, apparatus including a swallowable medical treatment device, which is configured to initially assume a swallowable contracted state, and which includes:

a gastric anchor, which initially assumes a contracted size, and which is configured to, upon coming in contact with stomach contents of a subject, expand sufficiently to prevent passage of the anchor through a pylorus of the subject even when the pylorus is in an open, relaxed state; and

a duodenal unit, which is configured to pass through the pylorus into a duodenum of the subject, and which is coupled to the gastric anchor such that the duodenal unit is held in the duodenum.

For some applications, the apparatus further includes a tether, which couples the duodenal unit to the gastric anchor, and has a length of between 1 cm and 20 cm.

For some applications, the duodenal unit includes one or more elongated members, each of which has a length of between 1 and 20 cm. For some applications, the elongated members are configured to expand upon coming in contact with a duodenal liquid.

For some applications, the gastric anchor is configured to serve as a pyloric plug, which is configured to at least partially block the pylorus. For some applications, the gastric anchor includes a duodenal plug component, which is configured to at least partially pass through the pylorus, and expand upon coming in contact with a duodenal liquid, so as to at least partially block the pylorus. For some applications, the duodenal unit is coupled to the gastric anchor via the duodenal plug component. For some applications, the duodenal unit includes one or more elongated members, which are coupled to the duodenal plug component.

For some applications, the gastric anchor includes a flexible sheet, which may, for example, have an area of less than 30 cm2. For some applications, the gastric anchor, when expanded, is bowl-shaped. For some applications, the gastric anchor includes a frame, which includes a plurality of ribs, to which the flexible sheet is coupled. For some applications, the flexible sheet has an area of less than 30 cm2. For some applications, the bowl-shaped gastric anchor is shaped so as to define a rim, and the gastric anchor further includes a band, which is coupled to the rim, and configured to prevent inversion of the bowl-shaped gastric anchor. For some applications, the band is biodegradable, such that, upon degrading, the band no longer prevents the inversion of the bowl-shaped gastric anchor.

For some applications, the gastric anchor is configured to define a passage therethrough, which has a size that varies after the anchor has expanded.

For some applications, the apparatus further includes a dissolvable enclosure that entirely surrounds the swallowable medical treatment device when the device initially assumes the swallowable contracted state.

There is still further provided, in accordance with an application of the present invention, apparatus including a swallowable medical treatment device, which is configured to initially assume a contracted state having a volume of less than 4 cm3, and which includes:

a gastric plug, which initially assumes a contracted size, and which is configured to, upon coming in contact with a liquid, expand to assume a bowl shape that defines a rim having a perimeter of between 3 cm and 12 cm; and

a duodenal unit, which is coupled to the gastric plug such that the duodenal unit is held between 1 cm and 20 cm from the gastric plug.

For some applications, the plug includes a flexible sheet. For some applications, the plug includes a frame, which includes a plurality of ribs, to which the flexible sheet is coupled. The flexible sheet may, for example, have an area of less than 30 cm2, and/or an area of at least 3 cm2.

For some applications, the plug further includes a band, which is coupled to the rim, and configured to prevent inversion of the bowl-shaped plug. For some applications, the band is biodegradable, such that, upon degrading, the band no longer prevents the inversion of the bowl-shaped gastric anchor.

For some applications, the apparatus further includes a tether, which couples the duodenal unit to the gastric anchor, and has a length of between 1 cm and 20 cm.

For some applications, the duodenal unit includes one or more elongated members, each of which has a length of between 1 and 20 cm. For some applications, the elongated members are configured to expand upon coming in contact with a duodenal liquid.

There is still further provided, in accordance with an application of the present invention, apparatus including a swallowable medical treatment device, which is configured to initially assume a contracted state having a volume of less than 4 cm3, and which includes:

a gastric anchor, which (a) includes a flexible sheet which initially is rolled to assume a contracted size, (b) is configured to, upon coming in contact with a liquid, unroll to assume an expanded size that is sufficient to prevent passage of the anchor through a round opening having a diameter of between 1 cm and 3 cm, and (c) is shaped so as to define a hole therethrough having a radius of at least 0.4 cm; and

a medical treatment component, which is coupled to the gastric anchor.

For some applications, the treatment component is configured to dispense a drug. For some applications, the treatment component includes an electrical stimulator. For some applications, the treatment component includes a bariatric sleeve.

For some applications, the flexible sheet is initially rolled around at least a portion of the medical treatment component.

For some applications, the flexible sheet has a length of between 20 and 40 mm, and a width of between 10 and 30 mm. For some applications, the flexible sheet, when initially rolled to assume the contracted size, has an outer diameter of between 10 and 20 mm.

For some applications, the flexible sheet is initially held rolled to assume the contracted size by one or more dissolvable elements.

For any of the applications described above, the apparatus may further include a dissolvable enclosure that entirely surrounds the swallowable medical treatment device when the flexible sheet is initially rolled to assume the contracted size.

For any of the applications described above, the opening may be a pylorus of a subject, the liquid may be stomach contents of the subject, the gastric anchor may be configured to, upon coming in contact with the stomach contents, unroll to assume the expanded size that is sufficient to prevent passage of the anchor through the pylorus, and the hole may be sized to allow chyme to pass to the pylorus.

There is additionally provided, in accordance with an application of the present invention, a method including:

receiving, by a subject, a swallowable medical treatment device in an initially contracted state, which device includes a gastric anchor having an initially contracted size, and a duodenal unit coupled to the gastric anchor; and

swallowing the treatment device by the subject, so that the anchor, upon coming in contact with stomach contents of the subject, expands sufficiently to prevent passage of the anchor through a pylorus of the subject, and the duodenal unit passes through the pylorus into a duodenum of the subject and is held in the duodenum by the anchor.

For some applications, receiving includes receiving the swallowable treatment device that further includes a tether, which couples the duodenal unit to the gastric anchor, and has a length of between 1 cm and 20 cm.

For some applications, receiving includes receiving the swallowable treatment device in which the duodenal unit is configured to dispense a drug.

For some applications, receiving includes receiving the swallowable treatment device in which the duodenal unit includes two or more duodenal stimulation electrodes that are configured to come in physical contact with a wall of the duodenum, and in which the treatment device further includes a power source and circuitry that is configured to drive the electrodes to apply an electrical current to the wall of the duodenum.

For some applications, receiving includes receiving the swallowable treatment device in which the duodenal unit includes a bariatric sleeve sized to allow chyme to pass therethrough without coming into contact with a wall of at least a portion of the duodenum.

For some applications, receiving includes receiving the swallowable treatment device in which the gastric anchor includes a flexible sheet which initially is rolled around at least a portion of the duodenal unit to assume the initially contracted size, and which is configured to prevent the passage of the anchor through the pylorus by unrolling upon coming in contact with the stomach contents.

For some applications, receiving includes receiving the swallowable treatment device in which the gastric anchor includes a flexible sheet which initially is rolled to assume the initially contracted size, and which is configured to prevent passage of the anchor through the pylorus by unrolling upon coming in contact with the stomach contents, which flexible sheet is shaped so as to define a passage therethrough. For some applications, the passage is shaped as a hole therethrough having a radius of at least 0.4 cm, through which chyme can pass to the pylorus.

For some applications, the gastric anchor is configured to serve as a pyloric plug, and swallowing includes swallowing the treatment device such that the pyloric plug at least partially blocks the pylorus. For some applications, the gastric anchor includes a duodenal plug component, which is configured to at least partially pass through the pylorus, and swallowing includes swallowing the treatment device such that the duodenal plug component at least partially passes through the pylorus, and expands upon coming in contact with a duodenal liquid, so as to at least partially block the pylorus. For some applications, the duodenal unit is coupled to the gastric anchor via the duodenal plug component. For some applications, the duodenal unit includes one or more elongated members, which are coupled to the duodenal plug component.

For some applications, receiving includes receiving the treatment device in which the plug includes a flexible sheet. For some applications, receiving includes receiving the treatment device in which the plug, when expanded, is bowl-shaped. For some applications, the plug includes a frame, which includes a plurality of ribs, to which the flexible sheet is coupled, and receiving the treatment device includes receiving the treatment device when the ribs are in a compressed state, and swallowing includes swallowing the treatment device such that the ribs expand sufficiently to prevent the passage of the plug through the pylorus.

For some applications, swallowing includes swallowing the treatment device such that the pyloric plug fully blocks the pylorus. For some applications, swallowing includes swallowing the treatment device such that the pyloric plug intermittently at least partially blocks the pylorus. For some applications, receiving includes receiving the swallowable treatment device in which the pyloric plug is configured to define a passage therethrough, which has a size that varies after the anchor has expanded. For some applications, receiving includes receiving the swallowable treatment device in which the pyloric plug is configured such that a size of the passage decreases in response to pulling on the pyloric plug by the duodenal unit. For some applications, receiving includes receiving the swallowable treatment device in which at least a portion of the plug is shaped so as to define a conical helix when in a resting state. For some applications, receiving includes receiving the swallowable treatment device that further includes a tether, which passes through at least a portion of the conical helix, and couples the duodenal unit to a vertex of the conical helix.

For some applications, receiving includes receiving the swallowable treatment device in which the gastric anchor, when expanded, is shaped as a sphere.

For some applications, receiving includes receiving the swallowable treatment device in which the duodenal unit includes one or more elongated members, each of which has a length of between 1 and 20 cm.

For some applications, receiving includes receiving the swallowable treatment device in which the elongated members are configured to expand upon coming in contact with a duodenal liquid.

There is yet additionally provided, in accordance with an application of the present invention, a method including:

receiving, by a subject, a swallowable treatment device in an initially contracted state, which includes (i) a gastric anchor, which includes a flexible sheet which initially is rolled to assume a contracted size, and which is shaped so as to define a hole therethrough having a radius of at least 0.4 cm, and (ii) a treatment component coupled to the gastric anchor; and

swallowing the treatment device by the subject, so that the anchor, upon coming in contact with stomach contents of the subject, unrolls sufficiently to prevent passage of the anchor through a pylorus of the subject, and to allow chyme to pass through the hole to the pylorus.

The present invention will be more fully understood from the following detailed description of applications thereof, taken together with the drawings, in which:

### BRIEF DESCRIPTION OF THE DRAWINGS

FIG. 1 is a schematic illustration of a swallowable medical treatment device in an initial contracted swallowable state, in accordance with an embodiment of the present invention;

FIG. 2 is a schematic illustration of the medical treatment device of FIG. 1 in an expanded state in a stomach of a subject, in accordance with an application of the present invention;

FIG. 3 is a schematic illustration of the medical treatment device of FIG. 1 in an anchored position, in accordance with an application of the present invention;

FIGS. 4 and 5 are schematic illustrations of an electrical stimulation duodenal unit of the medical treatment device of FIG. 1, in accordance with respective embodiments of the present invention;

FIG. 6 is a schematic illustration of a configuration of the duodenal unit of the medical treatment device of FIG. 1 including expandable electrodes, in accordance with an application of the present invention;

FIG. 7 is a schematic illustration of a bariatric sleeve duodenal unit of the medical treatment device of FIG. 1, in accordance with an application of the present invention;

FIG. 8 is a schematic illustration of the device of FIG. 7 in an initial contracted swallowable state, in accordance with an application of the present invention;

FIG. 9 is a schematic illustration of another configuration of the swallowable medical treatment device of FIG. 1 in the initial contracted swallowable state, in accordance with an application of the present invention;

FIGS. 10A and 10B are schematic illustrations of the treatment device of FIG. 9 in an expanded state in the stomach and in an anchored position, respectively, in accordance with an application of the present invention;

FIGS. 11A-C are schematic illustrations of the pyloric plug of the treatment device of FIG. 9 configured to define a variably-sized passage, in accordance with an application of the present invention;

FIG. 12 is a schematic illustration of another configuration of the pyloric plug of FIGS. 11A-C, in accordance with an application of the present invention;

FIGS. 13A-B and 13C are schematic illustrations of another configuration of the treatment device of FIG. 9 in an expanded state in the stomach and in an anchored position, respectively, in accordance with an application of the present invention;

FIGS. 14A-B and 14C are schematic illustrations of yet another configuration of the treatment device of FIG. 9 in an expanded state in the stomach and in an anchored position, respectively, in accordance with an application of the present invention;

FIGS. 14D-F are schematic illustrations of additional configurations of elongated members of the treatment device of FIG. 9, in accordance with respective applications of the present invention;

FIGS. 15A-B and 15C are schematic illustrations of still another configuration of the treatment device of FIG. 1 in an expanded state in the stomach and in an anchored position, respectively, in accordance with an application of the present invention;

FIGS. 16A-D are schematic illustrations of several configurations of the pyloric plug of FIGS. 15A-C, in accordance with respective applications of the present invention;

FIGS. 17A-B and 17C are schematic illustrations of another configuration of the pyloric plug of FIGS. 15A-C in an expanded state in the stomach and in an anchored position, respectively, in accordance with an application of the present invention;

FIGS. 18A-D are schematic illustrations of a configuration of the pyloric plug of FIGS. 15A-C anchored in the stomach and passing through the pylorus, in accordance with an application of the present invention; and

19A-B are schematic illustrations of another configuration of the swallowable medical treatment device in which the device serves as a pyloric plug, in accordance with an application of the present invention.

### DETAILED DESCRIPTION OF APPLICATIONS

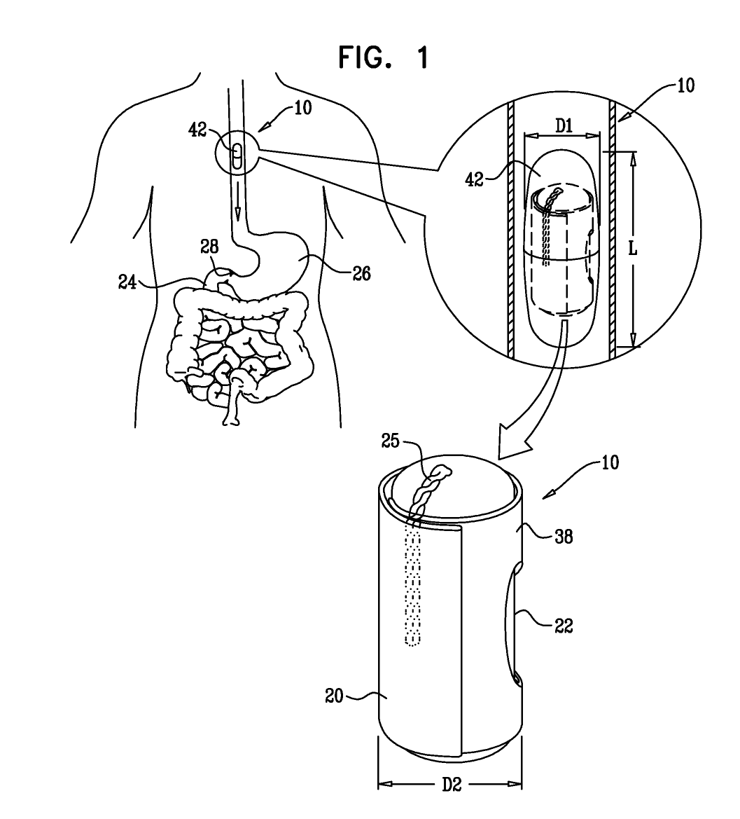

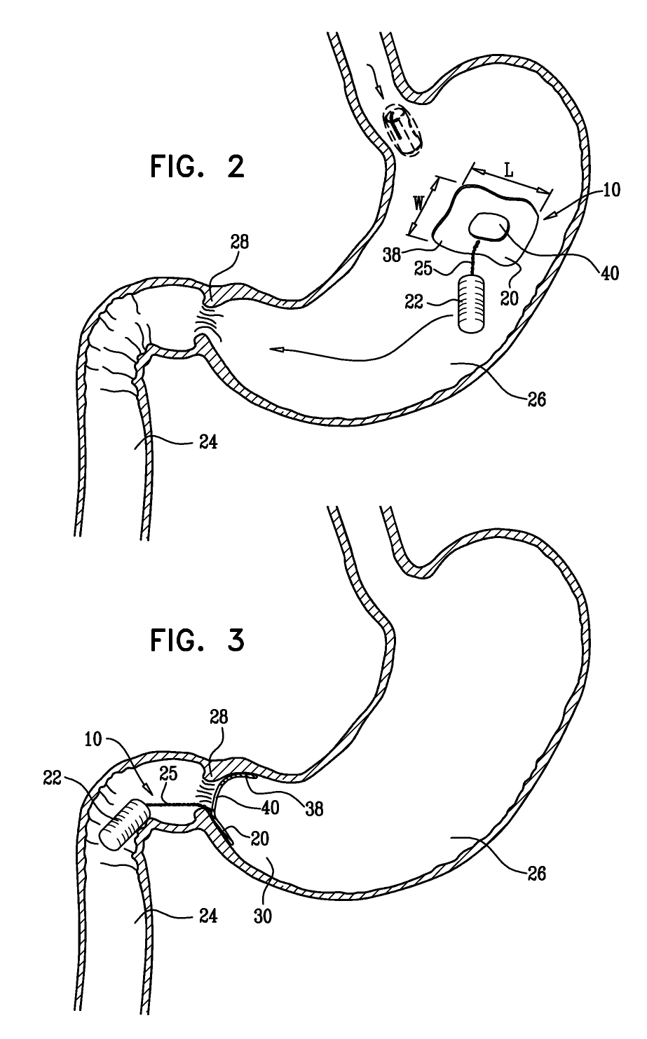

FIG. 1 is a schematic illustration of a swallowable medical treatment device 10 in an initial contracted swallowable state, in accordance with an embodiment of the present invention. Treatment device 10 comprises a gastric anchor 20, and, coupled to the anchor, a duodenal unit 22 configured to reside in a duodenum 24 of a subject. For some applications, the treatment device further comprises a tether 25 that couples the anchor to the duodenal unit.

Gastric anchor 20 initially assumes a contracted swallowable state, as shown in FIG. 1. In this configuration, treatment device 10 typically has a total volume (including enclosure 42, if provided, as described hereinbelow) of less than about 4 cm3, such as less than about 3 cm3, to readily allow swallowing by the subject. For some applications, when in the initial, contracted swallowable configuration, treatment device 10 has an outer diameter D1 (including enclosure 42, if provided, as described hereinbelow) of less than 15 mm, e.g., between about 7 and about 13 mm, and/or a total length L of less than 35 mm, such as between about 8 and about 30 mm. For some applications, duodenal unit 22 has a volume of at least 0.2 cc, or a volume of no more than 10 cc, or a volume of between 0.2 and 10 cc.

FIG. 2 is a schematic illustration of treatment device 10 in an expanded state in a stomach 26 of the subject, in accordance with an application of the present invention. After being swallowed, entering stomach 26, and coming in contact with stomach contents, anchor 20 expands, such as by unrolling, to prevent passage of the anchor through a pylorus 28 even when the pylorus is in an open, relaxed state. More generally, anchor 20 is configured to initially assume a contracted size, and, upon coming in contact with a liquid, to expand sufficiently to prevent passage of the anchor through a round opening having a diameter of between 1 cm and 3 cm. Alternatively, anchor 20 is anchored in the stomach using a technique other than expansion.

FIG. 3 is a schematic illustration of treatment device 10 in an anchored position, in accordance with an application of the present invention. After anchor 20 expands, gastric peristalsis positions treatment device 10 in an antrum 30 of stomach 26 in a vicinity of pylorus 28. Duodenal unit 22 is configured to arrive in the vicinity of pylorus 28 before anchor 20 arrives in the vicinity. For example, the duodenal unit may have a greater mass and/or density than the anchor, and/or be shaped to generate less resistance against the contents of the stomach (e.g., be rounded or smaller than the anchor). Duodenal unit 22 passes into duodenum 24 and is held by anchor 20 from passing further into the gastrointestinal (GI) tract. Typically, the duodenal unit is coupled to the gastric anchor such that the duodenal unit is held within about 1 cm to about 20 cm of the gastric anchor, such as within about 5 cm of the gastric anchor, e.g., within 2-5 cm of the gastric anchor. For applications in which treatment device 10 comprises tether 25, the tether holds duodenal unit 22 from passing further into the GI tract. Typically, the tether has a length of between about 1 cm and about 20 cm, such as between about 2 cm and about 5 cm, such that duodenal unit 22 is held in duodenum 24.

At least a portion of anchor 20 eventually biodegrades in the stomach, causing the anchor to break down or break apart into smaller pieces, and the entire device to be evacuated through the GI tract by peristalsis (not shown). For some applications, the anchor is configured to biodegrade between about 1 and about 24 hours after the device is swallowed, such as between about 1 and about 8 hours after the device is swallowed.

For some applications, gastric anchor 20 comprises a flexible sheet 38 sized to prevent passage of the anchor through the pylorus, as shown in FIGS. 1-3. For some applications, sheet 38 is shaped so as to define a passage 40 therethrough, such as a hole (e.g., a central passage), through which chyme can pass to the pylorus and the duodenum. For some applications, the passage (e.g., hole) is larger than the opening of pylorus 28 when open, and large enough to allow passage through passage 40 (e.g., hole) of duodenal unit 22. For applications in which the passage is a hole, the hole typically has a radius of between about 0.25 and about 2 cm, such as between about 0.5 and about 1 cm. Before the device is swallowed, sheet 38 is rolled to assume a contracted, swallowable size, as shown in FIG. 1. Upon arriving in the stomach, as shown in FIG. 2, the sheet unrolls and is positioned in antrum 30 by gastric peristalsis, as shown in FIG. 3. (The duodenal unit sometimes passes through the pylorus before the anchor settles near the pylorus, and sometimes passes through passage 40 (e.g., hole) after the anchor settles near the pylorus.)

For some applications, sheet 38 is initially rolled around at least a portion of the duodenal unit, as shown in FIG. 1. The sheet may be held wrapped around the duodenal unit by an adhesive, such as for applications in which dissolvable enclosure 42 is not provided, or even for applications in which the enclosure is provided. Alternatively, the sheet is initially positioned longitudinally or laterally adjacent to duodenal unit 22, and the sheet and unit are removably coupled to one another, such that they come decoupled upon exposure to the contents of the stomach (configurations not shown). Further alternatively, the sheet and duodenal unit are initially coupled together only by tether 25 (configuration not shown). For some applications, the sheet is initially held in the rolled position by one or more dissolvable elements, such as one or more dissolvable rings placed around the rolled sheet (e.g., comprising gelatin), or a dissolvable glue that binds the outermost edge of the sheet to a more inner portion of the sheet. These dissolvable elements dissolve once the device reaches stomach 26. Alternatively or additionally, the sheet is initially held in the rolled position by a dissolvable capsule or coating, as described hereinbelow.

For some applications, sheet 38 has a length L of between about 20 and about 40 mm, such as about 25 mm, and a width of between about 10 and about 30 mm, such as about 25 mm, as indicated in FIG. 2. Typically, the width of sheet 38 is approximately equal to the length of duodenal unit 22. For some applications, when the sheet assumes its initial rolled position, as shown in FIG. 1, the sheet has an outer diameter D2 of between about 10 and about 20 mm.

For some applications in which anchor 20 comprises sheet 38 defining passage 40 (e.g., hole), treatment device 10 does not comprise duodenal unit 22. Instead, the anchor is coupled to another medical treatment component that remains in the stomach with the anchor. For example, the treatment component may comprise a drug (e.g., a slow-release drug), an electrical stimulator configured to apply electrical stimulation to the stomach, or both the drug and the electrical stimulator. For example, the electrical stimulator may apply the electrical stimulation at between 5 and 7 mA, at a frequency of between 5 and 40 Hz (e.g., 30 Hz), optionally in pulse trains (e.g., 5 second on periods alternating with 2.5 second off periods), for example to generate peristalsis.

Alternatively, gastric anchor 20 uses other chemical and/or mechanical techniques for expansion. For example, the anchor may comprise a material (e.g., a gel, a sponge, or bicarbonate) that swells upon contact with the liquid contents of the stomach, and/or a balloon or a sponge that fills with a gas upon contact with the liquid contents of the stomach. Alternatively, the anchor may comprise one or more mechanical elements that are initially held in a compressed position, and expand, e.g., unfold (e.g., like an accordion), upon being released when the device reaches the stomach. For some applications, expansion techniques are used that are described in the above-mentioned article by Klausner E A et al. and/or the other references mentioned hereinabove in the Background of the Application section.

For some applications, treatment device 10 comprises a dissolvable enclosure 42 that entirely surrounds device 10 when the device initially assumes its contracted swallowable state, thereby encapsulating or coating the device, such as shown in FIG. 1. For example, dissolvable enclosure 42 may comprise a hard- or soft-shelled capsule or coating, e.g., comprising gelatin or another water-soluble material. The enclosure facilitates safe and easy swallowing of the device, and dissolves once the device reaches stomach 26. In addition, the enclosure may help prevent expansion of the device before it reaches the stomach.

In some embodiments of the present invention, the duodenal unit is configured to dispense a drug, such as a drug for treating diabetes and/or obesity. For some applications, the duodenal unit comprises a conventional drug pill comprising the drug. The pill may comprise, for example, a capsule. Alternatively, the duodenal unit may comprise a slow-release reservoir that slowly releases the drug into the duodenum. For some applications, anchor 20 is alternatively or additionally coated with a drug (either the same drug as or a different drug from that of the duodenal unit).

For some applications, the drug comprises one or more of the following drugs for treating diabetes:
a sulfonylurea, such as Dymelor, Diabinese, Orinase, Tolinase, Glucotrol, Glucotrol XL, DiaBeta, Micronase, Glynase PresTab, and Amaryl;
a biguanide, such as metformin (Glucophage, Glucophage XR, Riomet, Fortamet and Glumetza);
a thiazolidinedione, such as Actos and Avandia;
an alpha-glucosidase inhibitor, such as Precose and Glyset;
a meglitinide, such as Prandin and Starlix;
a dipeptidyl peptidase IV (DPP-IV) inhibitor, such as Januvia; and
a combination therapy drug, such as Glucovance (which combines glyburide (a sulfonylurea) and metformin), Metaglip (which combines glipizide (a sulfonylurea) and metformin), and Avandamet (which combines metformin and rosiglitazone (Avandia)).

For some applications, the drug comprises one or more of the following drugs for treating obesity and/or promoting weight loss:
dexfenfluramine (e.g., Redux);
diethylpropion (e.g., Tenuate, Tenuate dospan);
fenfluramine (e.g., Pondimin);
mazindol (e.g., Sanorex, Mazanor);
orlistat (e.g., Xenical);
phendimetrazine (e.g., Bontril, Plegine, Prelu-2, X-Trozine);
phentermine (e.g., Adipex-P, Fastin, Ionamin, Oby-trim); and
sibutramine (e.g., Meridia).

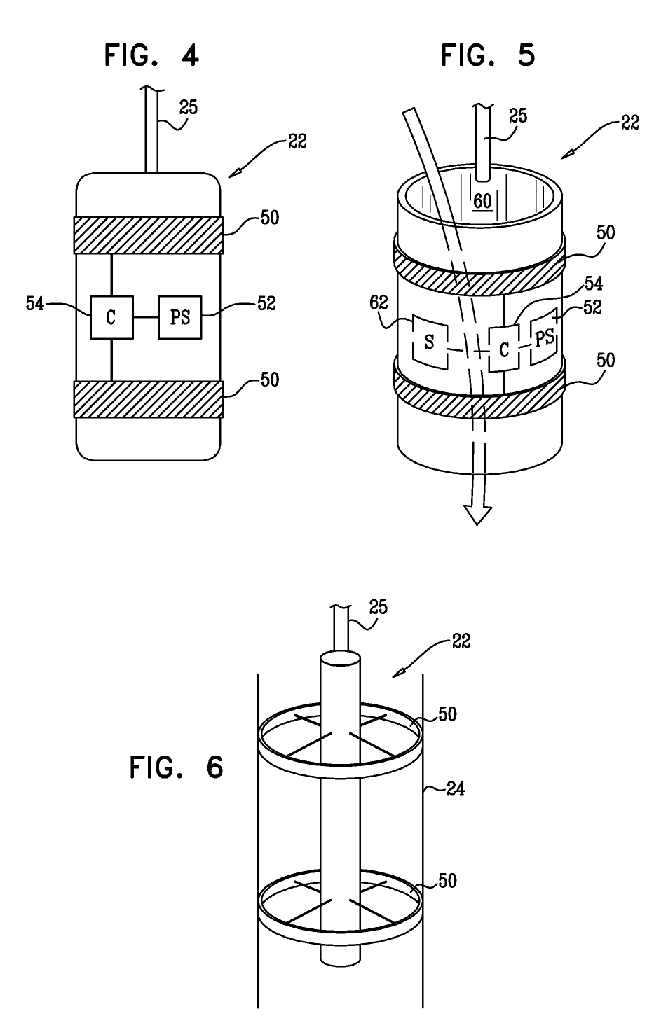

Reference is made to FIG. 4, which is a schematic illustration of an electrical stimulation application of duodenal unit 22, in accordance with an application of the present invention. In this embodiment, duodenal unit 22 comprises two or more duodenal stimulation electrodes 50 that are configured to come in physical contact with the wall of duodenum 24. For some applications, one or more of the electrodes wrap around the outer surface of the duodenal unit, as shown in FIG. 4. Alternatively or additionally, one or more of the electrodes are oriented along the length of the duodenal unit (configuration not shown). The treatment device comprises a power source 52, such as a battery, and circuitry 54 that is configured to drive electrodes 50 to apply an electrical current to the wall of the duodenum.

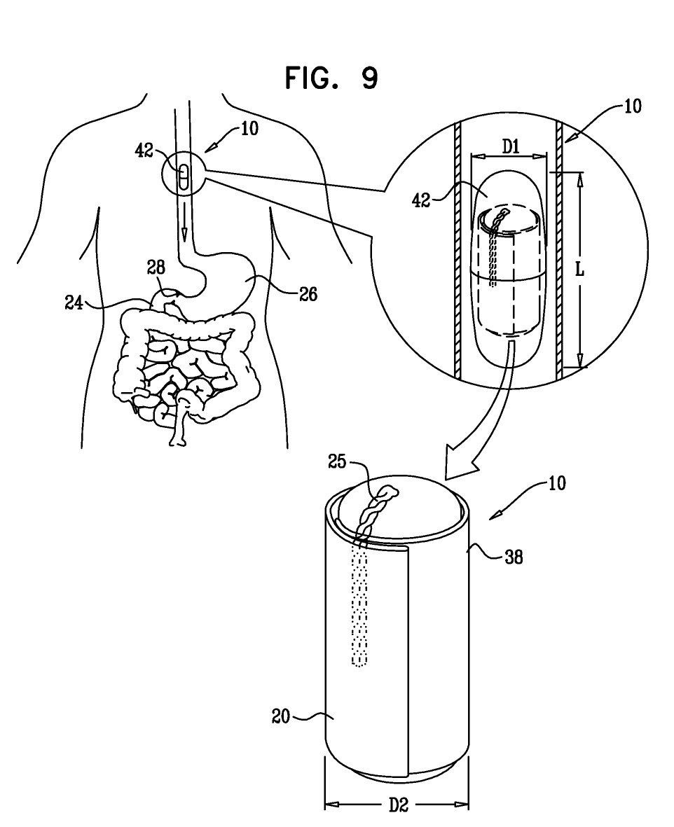

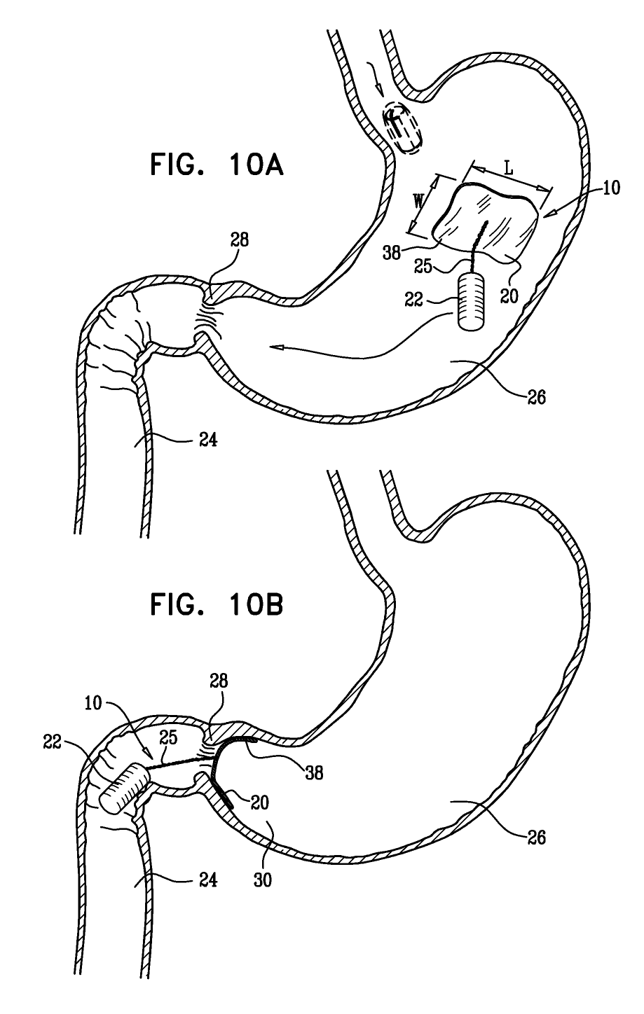

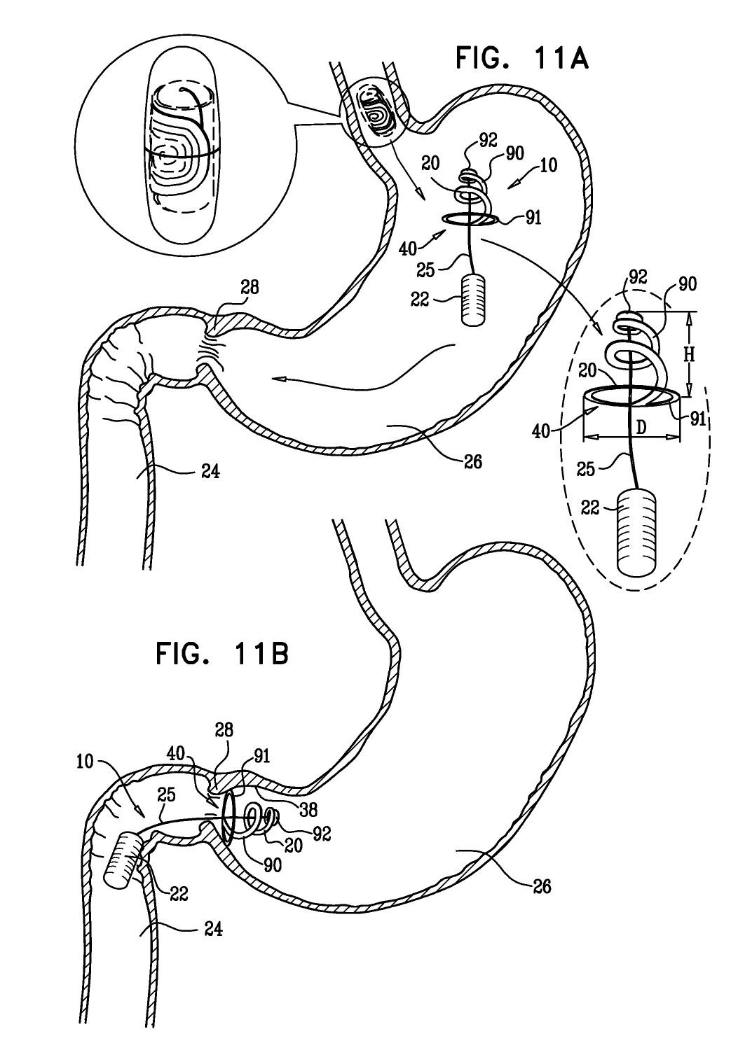

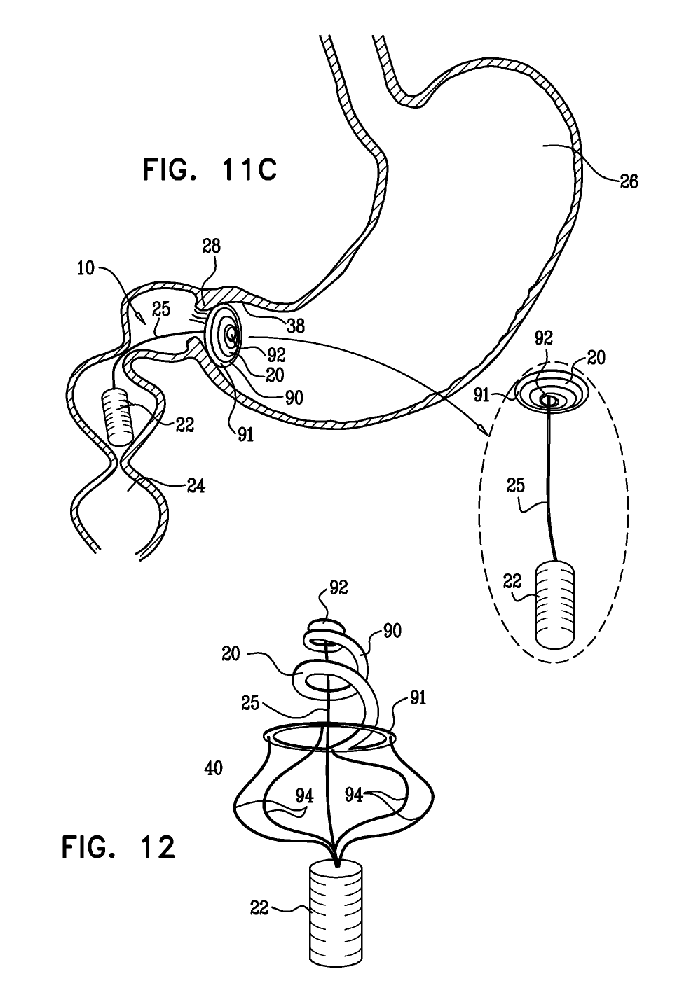

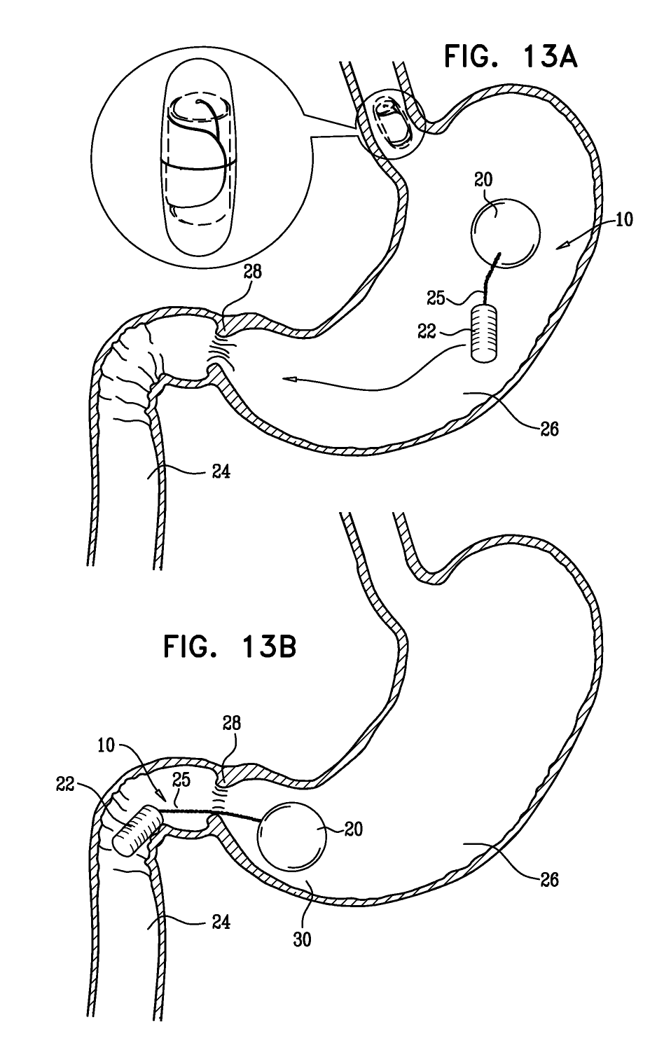

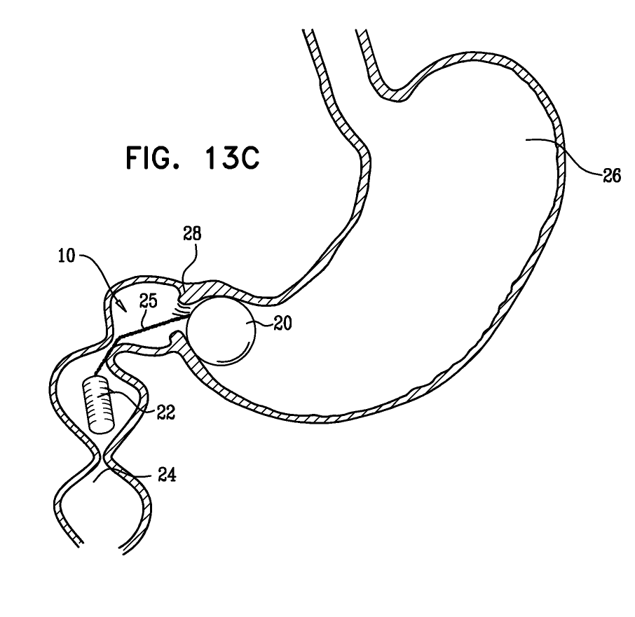

For some applications, circuitry 54 configures the current to induce and/or increase a rate of peristalsis in the duodenum, and/or induce migrating motor complex (MMC) in the duodenum. As a result, the residence time of absorbable food calories in the duodenum is reduced, as is glucose uptake and other forms of calorie uptake. Alternatively or additionally, such an increased rate of peristalsis may increase the strength with which duodenal unit 22 pulls on the pyloric plug, for the applications described hereinbelow with reference to FIG. 9, 10A-B, 11A-C, 12, 13A-C, or 15A-C.

For some applications, circuitry 54 is configured to stimulate the vagus nerve, thereby generating satiety-related signals that travel to the brain and cause satiety (see, for example, the above-mentioned U.S. Pat. No. 5,188,104 to Wernicke et al., which is incorporated herein by reference, and which describes vagal stimulation techniques for inducing satiety). For example, parameters described in the following paragraph may be used for stimulating the vagus nerve. For some applications, tether 25 has a length of up to 10 cm, which holds duodenal unit 22 in the duodenum near the pylorus near a branch of the vagus nerve.

For some applications, circuitry 54 configures the current to have an amplitude of between 2 and 10 mA, e.g., between 4 and 6 mA, such as 5 mA. For some applications, circuitry 54 applies the current in a series of pulses, each of which has a duration of between 0.1 and 10 milliseconds, e.g., between 0.5 and 2 milliseconds (such as 1 millisecond), or between 2 and 7 milliseconds (such as 5 milliseconds). For some applications, circuitry 54 applies the current in a plurality of series of pulses, which series are separated by periods during which the current is not applied. For some applications, the circuitry applies the pulses at a frequency of between 10 and 100 Hz, such as between 15 and 30 Hz (e.g., 20 Hz), between 25 and 75 Hz (e.g., 50 Hz), or between 75 and 125 Hz (e.g., 100 Hz). For some applications, circuitry 54 applies the current intermittently during stimulation periods alternating with non-stimulation periods. For example, the stimulation periods may have a duration of about an hour, and the non-stimulations periods may have a duration of about 30 minutes, and the device may apply stimulation for a total of about 8 hours until the device biodegrades. For some applications, the circuitry applies the pulses as square pulses. For some applications, the circuitry configures the pulses to be biphasic (e.g., each phase may have a duration equal to half of the pulse duration). For some applications, the circuitry applies the pulses in a train, e.g., having “on” periods (e.g., each of which having a duration of about two seconds) alternating with “off” periods (e.g., each of which having a duration of between about 3 and about 8 seconds).

For some applications, circuitry 54 is configured to intermittently drive electrodes 50 to apply the current. For example, the circuitry may drive the electrodes to apply the current during activation periods each of which has a duration of between about five and about fifteen minutes (e.g., about ten minutes), alternating with non-stimulation periods each of which has a duration of between about 30 and about 60 minutes.

For some applications, circuitry 54 is configured to wait a certain period of time after the duodenal unit enters the duodenum before driving the electrodes to induce peristalsis, thereby allowing time for food to enter the duodenum from the stomach. The subject may swallow treatment device 10 before beginning a meal. As appropriate, techniques described in the above-cited article to Sun et al. may be adapted for use in this application for stimulating the duodenum.

For some applications, duodenal unit 22 comprises power source 52 and/or circuitry 54, while for other applications, gastric anchor 20 comprises the power source and/or circuitry, in which case tether 25 may comprise one or more wires to convey the current to the duodenal unit, or the device is configured to wirelessly transmit power from the anchor to the duodenal unit. For some applications, duodenal unit 22 comprises a coating, such as an enteric coating.

FIG. 5 is a schematic illustration of another configuration of duodenal unit 22, in accordance with an application of the present invention. Other than as described below, this configuration is similar to the configuration described hereinabove with reference to FIG. 4. In this configuration, the duodenal unit is shaped so as to define a passage 60 therethrough, through which chyme can pass. For some applications, the duodenal unit is flexible, in order to accommodate peristaltic waves of the duodenum. For some applications, the gastric anchor is initially rolled up inside passage 60 of the duodenal unit.

For some applications, duodenal unit 22 comprises a sensor 62, which is configured to detect the passage of chyme through passage 60 or past the unit (such as for applications in which the unit is not shaped so as to define passage 60), and/or opening of pylorus 28. Circuitry 54 is configured to drive electrodes 50 to apply the current responsively to detection of chyme passage by the sensor (e.g., upon detection, or a certain amount of time after detection), and to cease driving the electrodes when chyme passage is no longer detected or the pylorus closes, or after a certain period of time. This regulated application of current may conserve power, and/or avoid any undesirable effects of excessive electrical stimulation of the duodenum. For some applications, sensor 62 detects opening of the pylorus by electromyographic (EMG) analysis of physiological electrical activity sensed by an electrode on the pylorus. Techniques for identifying a change in state of a muscle using EMG analysis are known in the art. Alternatively, other sensors adapted to sense pyloric opening and closing may be used, such as an acceleration sensor, a strain gauge, or an ultrasound sensor.

Alternatively or additionally, induction of the peristalsis or MMC is initiated in response to a detection of the occurrence of segmentation of the duodenum; the induced peristalsis or MMC typically terminates the segmentation process. For some applications, segmentation is detected responsively to a pattern of electrical activity along the duodenum that is measured by electrodes 50 and analyzed by circuitry 54. Alternatively, induction of the peristalsis or MMC is practiced not in response to any sensed event. For example, the peristalsis or MMC may be artificially initiated for a certain amount of time during one or more periods every day. For some applications, induction of the peristalsis or MMC is not performed when the subject is asleep.

Alternatively or additionally, treatment device 10 (either gastric anchor 20 or duodenal unit 22) comprises an eating sensor (e.g., a swallowing sensor), which is configured to generate a signal indicative of eating by the subject. Circuitry 54 is configured to drive electrodes 50 to apply the current responsively to the sensing of eating. Alternatively, the circuitry drives the electrodes to apply the current not responsively to sensing of eating.

For some applications, at least a portion of duodenal unit 22 is biodegradable, such that the duodenal unit eventually breaks down and is evacuated through the GI tract by peristalsis. Alternatively, the duodenal unit is not configured to be biodegradable, and is evacuated intact through the GI tract by peristalsis when anchor 20 breaks down, as described hereinabove with reference to FIG. 3.

FIG. 6 is a schematic illustration of a configuration of duodenal unit 22 in which electrodes 50 are expandable, in accordance with an application of the present invention. In this configuration, electrodes 50 are configured to expand to come in contact with the wall of duodenum 24, such as by elastic components, e.g., springs. Typically, the diameter of the body of duodenal unit 22 is sized to allow chyme to pass between the body and the duodenal wall.

For some applications, device 10 does not comprise gastric anchor 20 or tether 25. Instead, electrical stimulation duodenal unit 22 comprises a mucoadhesive applied to an external surface of the unit. The mucoadhesive causes the unit to adhere partially or completely to the wall of the duodenum, thereby slowing down or stopping motion of the unit in the duodenum for a period of time. For some applications, the unit comprises an enteric coating that coats the mucoadhesive, and is configured to dissolve in the duodenum, thereby preserving the mucoadhesive until the unit arrives in the duodenum.

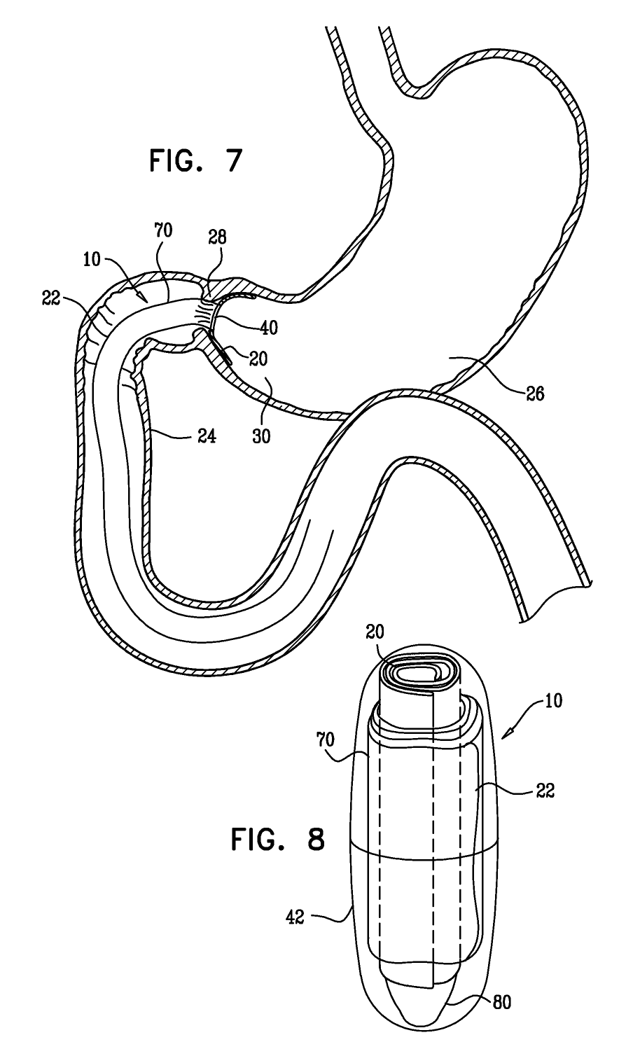

Reference is made to FIG. 7, which is a schematic illustration of an application in which duodenal unit 22 comprises a bariatric sleeve 70, in accordance with an application of the present invention. Bariatric sleeve 70 is sized to allow chyme to pass therethrough without coming into contact with the wall of at least a portion of duodenum 24. Such bypassing of the duodenum reduces absorption of nutrients and calories. Optionally, the sleeve is long enough to additionally bypass a portion of the jejunum. The sleeve is typically biodegradable, such that after a period of time the sleeve degrades and is evacuated through the GI tract by peristalsis. The length of the sleeve is typically between about 5 cm and about 30 cm, and may be selected for each subject depending upon the weight loss that is desired to be induced by the sleeve. The diameter of the sleeve is typically between about 10 and about 30 mm.

For some applications, as shown in FIG. 7, a proximal end of sleeve 70 is directly coupled to anchor 20 such that passage 40 (e.g., hole) of anchor 20 opens directly into the lumen of the sleeve. In other words, the proximal end of the sleeve is coupled to the anchor surrounding the passage (e.g., hole). For these applications, treatment device 10 typically does not comprise tether 25. The anchor and sleeve are typically biodegradable, or comprise a plurality of parts that separate over time, allowing the anchor and sleeve to pass through the GI tract.

Reference is made to FIG. 8, which is a schematic illustration of device 10 in an initial contracted swallowable state, in accordance with an application of the present invention. Before device 10 is swallowed by the subject, sleeve 70 and gastric anchor 20 assume initial contracted positions. For some applications, the sleeve is rolled around the contracted anchor (which, for applications in which the anchor comprises sheet 38, as shown in FIG. 8, is also rolled). Alternatively, sheet 38 is rolled around the sleeve (configuration not shown). As mentioned above, for some applications, device 10 comprises dissolvable enclosure 42 that entirely surrounds device 10 when the device initially assumes its contracted swallowable state, thereby encapsulating or coating the device.

Upon exposure to the contents of the stomach, the sleeve and anchor unroll. Gastric peristalsis moves the sleeve into the duodenum, where duodenal peristalsis extends the sleeve along the duodenum.

For some applications, the distal end of the sleeve is initially shaped to have a rounded tip 80 (e.g., bullet-shaped), which facilitates passage through the pylorus. After passing through the pylorus, the tip dissolves, allowing chyme to pass through the sleeve. Alternatively, for some applications, the distal end of the sleeve comprises a plug that facilitates passage through the pylorus. After the distal end of the sheet with the plug passes through the pylorus, the plug dissolves, allowing chyme to pass through the sleeve. Alternatively, the plug is configured to dissolve more slowly. Duodenal peristalsis naturally pulls the plug more than it pulls the sleeve, thereby causing the plug and distal end of the sleeve to be positioned more distally in the duodenum than is the sleeve. After the sleeve is extended in the duodenum, the plug dissolves.

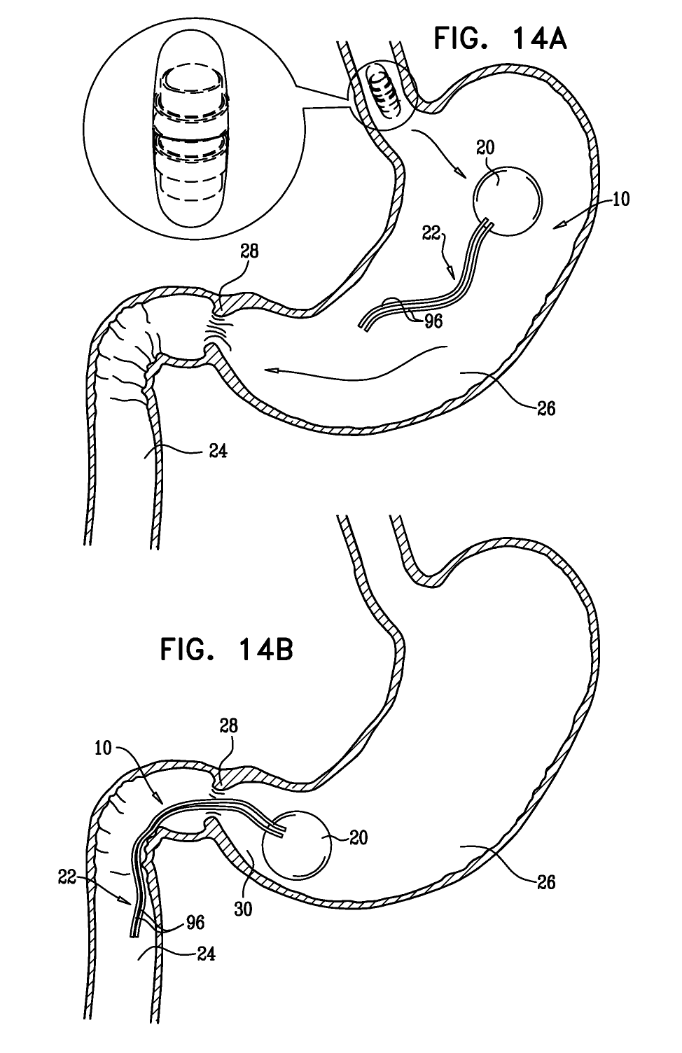

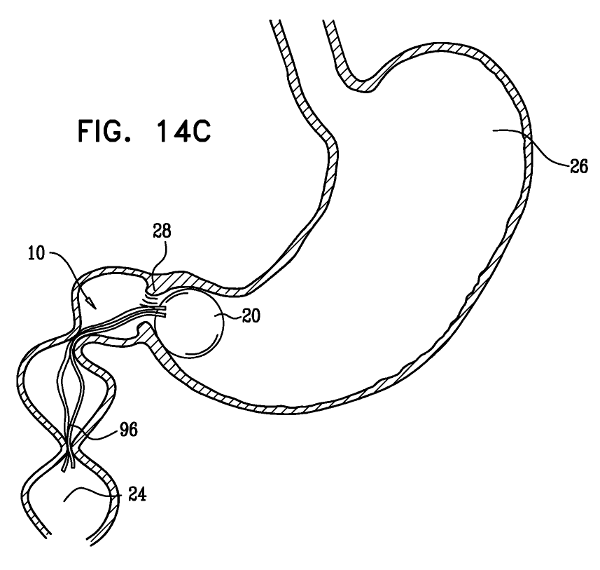

Reference is now made to FIG. 9, which is a schematic illustration of another configuration of swallowable medical treatment device 10 in the initial contracted swallowable state, in accordance with an application of the present invention. In this configuration, gastric anchor 20 is configured to serve as a pyloric plug, which is configured to at least partially block pylorus 28 upon being pulled toward the duodenum by duodenal unit 22. To this end, for some applications, as shown in FIG. 9 (and FIGS. 10A-B, 13A-C, 14A-C, 14D-F, 15A-C, 16A-D, 17A-C, 18A-D, and 19A-B), anchor 20 is not shaped so as to define a passage therethrough, and thus may fully or nearly fully block the pylorus at least a portion of the time that the anchor is in the stomach. Alternatively, anchor 20 is shaped so as to define a passage (e.g., a hole) that partially occludes pylorus 28, and is thus generally smaller than the orifice of the pylorus (configuration not shown). For applications in which the anchor is shaped so as to define the hole, the hole typically has a diameter of between 4 and 20 mm, such as between 4 and 8 cm.

Typically, the pyloric plug described with reference to FIGS. 9, 10A-B, 13A-C, 14A-C, 14D-F, 15A-C, 16A-D, 17A-C, and 18A-D partially or fully occludes the pylorus by covering the pylorus, such as by coming in contact with the wall of the antrum surrounding the pylorus, but without being inserted into the pylorus. Alternatively, the pyloric plug is at least partially inserted into the pylorus, for example as described hereinbelow with reference to FIGS. 19A-B.

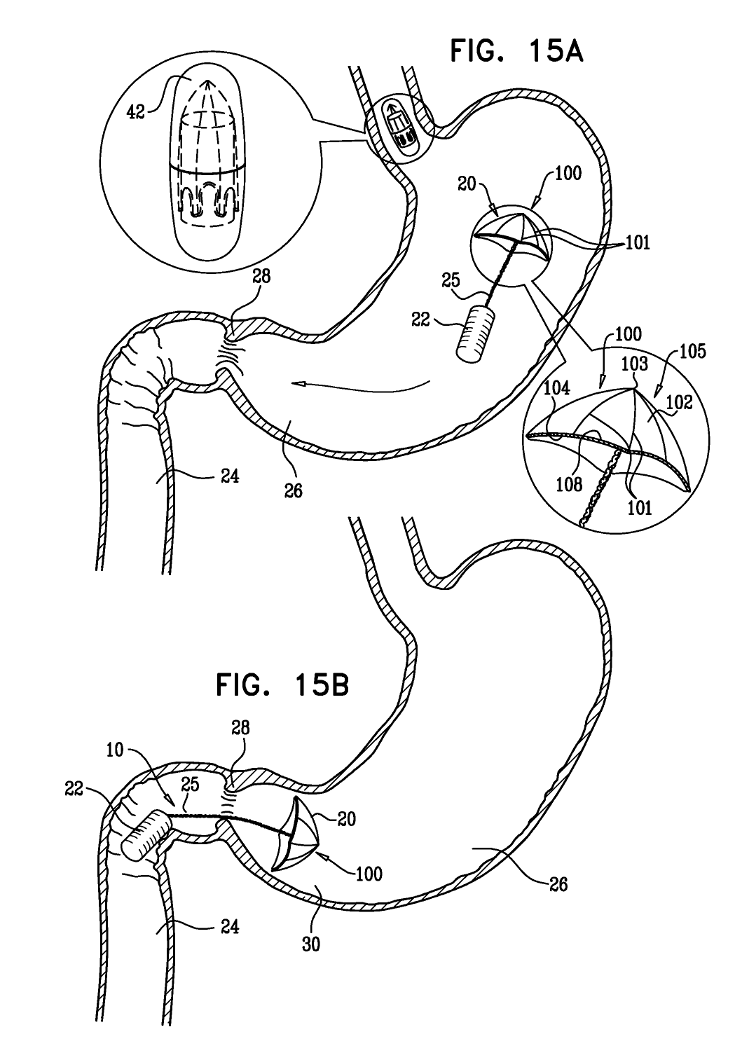

In the configuration described with reference to FIG. 9, gastric anchor may comprise flexible sheet 38, as described hereinabove with reference to FIGS. 1-3, or any of the other anchor configurations described herein (e.g., accordion or balloon), including the configuration described hereinbelow with reference to FIGS. 12A-B.

FIG. 10A is a schematic illustration of treatment device 10 in an expanded state in stomach 26, in accordance with an application of the present invention. After being swallowed, entering stomach 26, and coming in contact with stomach contents, anchor 20 expands, such as by unrolling, to prevent passage of the anchor through pylorus 28 even when the pylorus is in an open, relaxed state.

FIG. 10B is a schematic illustration of treatment device 10 in an anchored position, in accordance with an application of the present invention. Peristalsis advances treatment device 10 toward pylorus 28, and duodenal unit 22 into duodenum 24. Peristalsis in the duodenum advances the duodenal unit in the duodenum, causing the duodenal unit to pull the pyloric plug (such as via tether 25) toward the pylorus, until the pyloric plug at least partially (e.g., fully) blocks pylorus 28. Duodenal unit 22 is prevented by gastric anchor 20 from passing further into the GI tract.

Such a partial or full blockage of the pylorus induces a sensation of satiety, e.g., by slowing the passage of chyme from the stomach. The device thus may slow a rise in blood sugar during and after eating food. The device is thus useful for treating conditions such as obesity and diabetes.

Typically, use of this configuration of treatment device 10 results in intermittent, alternating periods of peristalsis in the duodenum, and corresponding periods of full or partial blocking of the pylorus. During periods in which chyme is not in the duodenum, and thus peristalsis does not occur, the duodenal unit does not hold the pyloric plug against the pylorus. Natural muscular activity of the stomach moves the pyloric plug away from the pylorus, allowing chyme to pass through the pylorus. Passage of chyme through the pylorus into the duodenum causes duodenal peristalsis, which causes the duodenal unit to pull the pyloric plug against the pylorus, and/or against the wall of the antrum surrounding the pylorus. This opened/closed cycling of the pylorus results in slow release of the chyme from the stomach into the duodenum.

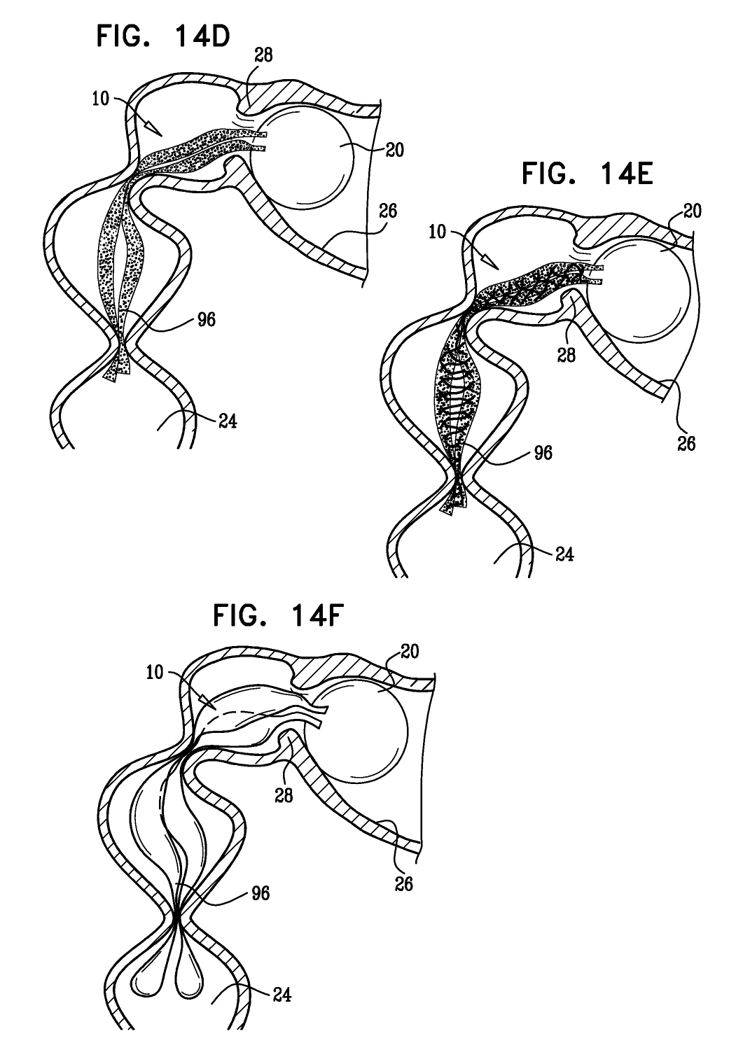

Reference is made to FIGS. 11A-C, which are schematic illustrations of the pyloric plug of treatment device 10 configured to define a variably-sized passage 40, in accordance with an application of the present invention. In this configuration, the pyloric plug is configured to define passage 40 therethrough, and is configured such that a size of the passage varies while the anchor is in its expanded state. In this configuration, the pyloric plug thus serves as a valve that regulates the passage of chyme from the stomach into the duodenum. For some applications, the size of the passage decreases in response to greater pulling on the plug by the duodenal unit. Thus, when chyme is present in the duodenum, duodenal peristalsis pulls on the duodenal unit, causing a reduction in the size of the passage, and a reduction of the amount of chyme that passes through the pylorus. As chyme passes out of the duodenum, peristalsis in the duodenum decreases, the duodenal unit pulls less on the duodenal unit, and the size of the passage increases. For some applications, pulling by the duodenal unit may close the passage entirely (i.e., the size of the passage is zero), such that the pyloric valve completely blocks the passage of chyme through the pylorus.

FIGS. 11A-C show one particular configuration of the pyloric plug defining a variably-sized passage. Other valve configurations will be evident to those skilled in the art who have read the present application, and are within the scope of the present invention. For example, the pyloric plug may comprise various combinations of springs, flexible and/or elastic materials, flaps, and other elements, arranged such that pulling on at least a portion of these elements by the duodenal unit changes a size of a passage through the pyloric plug.

In the specific configuration shown in FIGS. 11A-C, at least a portion of the pyloric plug comprises a curved strip of elastic material shaped as a conical helix 90 when the pyloric plug is in its resting state (i.e., duodenal unit 22 is not pulling on the plug). Tether 25 passes through at least a portion of the helix (typically, including a base 91 of the helix) and couples duodenal unit 22 to a vertex 92 of the conical helix, as best seen in FIGS. 11A and 11B.

As shown in FIG. 11A, device 10 is swallowed in a contracted state. After being swallowed, entering stomach 26, and coming in contact with stomach contents, the anchor 20 expands sufficiently to prevent passage of the anchor through a round opening having a diameter of between 1 cm and 3 cm, typically the pylorus. As shown in FIG. 11B, peristalsis advances treatment device 10 toward pylorus 28, and duodenal unit 22 into duodenum 24. As shown in FIG. 11C, duodenal peristalsis advances the duodenal unit distally in the duodenum. When, as a result of this advancement, the duodenal unit pulls the tether, the tether pulls the vertex toward the base of the helix, thereby at least partially collapsing the helix, and reducing a size of passage 40 therethrough. As shown in FIG. 11C, such pulling sometimes entirely closes the passage (i.e., the size of the passage is zero).

For some applications, conical helix 90 is elliptical, rather than circular; alternatively, the helix may be polygonal. For some applications, base 91 has a diameter D of at least 3 cm, or a diameter of no more than 6 cm, or a diameter of between 2 and 6 cm. For some applications, a height H of the pyloric plug when in its most open, resting state (i.e., a distance between vertex 92 and a plane defined by base 91) is at least 1 cm, or no more than 10 cm, or between 1 and 10 cm, e.g., between 2 and 7 cm. For some applications, base 91 defines a closed shape, e.g., corresponding to the shape of the helix, such as an ellipse (e.g., a circle), or a polygon.

FIG. 12 is a schematic illustration of another configuration of the helical pyloric plug, in accordance with an application of the present invention. In this configuration, duodenal unit 22 is coupled to the pyloric plug (typically to base 91 thereof) by one or more alignment cords 94 (e.g., one, two, three, four, or more than four), in addition to tether 25. Alignment cords 94 help align the pyloric plug in the stomach such that the base is oriented toward the pyloric valve. As the duodenal unit advances through the duodenum, the duodenal unit pulls the alignment cords, which in turn pull the base of the pyloric plug toward the pylorus. For some applications, each of alignment cords 94 has a length equal to between 80% and 120% (typically 100%) of a length of tether 25.

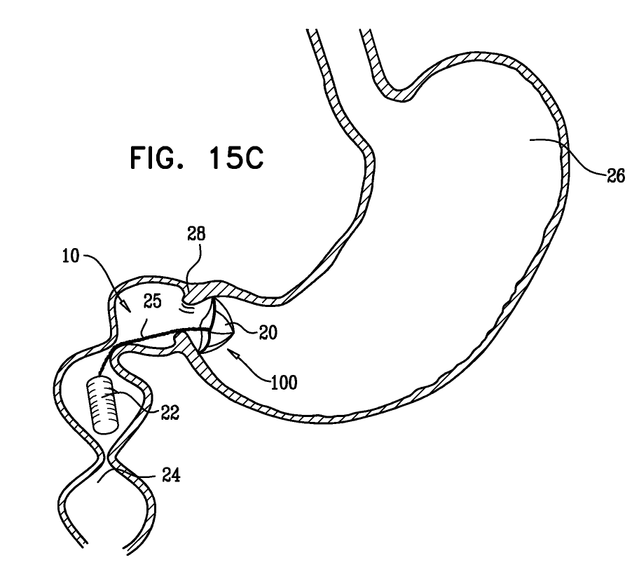

FIGS. 13A-B and 13C are schematic illustrations of another configuration of device 10 in an expanded state in the stomach and in an anchored position, respectively, in accordance with an application of the present invention. In this configuration, gastric anchor 20, when expanded, is shaped as a sphere, or another three-dimensional shape, or, alternatively, as a generally flat shape (e.g., as shown in FIG. 10A). As shown in FIG. 13A, and as mentioned above, the gastric anchor is swallowed in a contracted state. After being swallowed, entering stomach 26, and coming in contact with stomach contents, anchor 20 expands, to prevent passage of the anchor through pylorus 28 even when the pylorus is in an open, relaxed state. More generally, anchor 20 is configured to initially assume a contracted size, and, upon coming in contact with a liquid, to expand sufficiently to prevent passage of the anchor through a round opening having a diameter of between 1 cm and 3 cm. For example, the anchor may comprise a material (e.g., a gel, a sponge, or bicarbonate) that swells upon contact with the liquid contents of the stomach, and/or a balloon or a sponge that fills with a gas upon contact with the liquid contents of the stomach.

For some applications, upon expansion anchor 20 assumes a three-dimensional shape other than a sphere, such as a polyhedron. More generally, anchor 20 may comprise any structure that assumes contracted and expanded states. For some applications, when in its expanded state, the anchor has a greatest cross-section of at least 2 cm, typically at least 3 cm, to prevent passage through the pylorus.

As shown in FIG. 13B, peristalsis advances treatment device 10 toward pylorus 28, and duodenal unit 22 into duodenum 24. As shown in FIG. 13C, duodenal peristalsis advances the duodenal unit distally in the duodenum. As a result of this advancement, the duodenal unit pulls the pyloric plug (such as via tether 25) toward the pylorus, such that the plug at least partially blocks the pylorus, such as entirely blocks the pylorus.

For some applications, this configuration of treatment device 10 results in intermittent, alternating periods of peristalsis in the duodenum, and corresponding periods of full or partial blocking of the pylorus. During periods in which chyme is not in the duodenum, and thus peristalsis does not occur, duodenal unit 22 does not hold the pyloric plug against the pylorus. Natural muscular activity of the stomach moves the pyloric plug away from the pylorus, again assuming the position shown by way of example in FIG. 13B, and allowing chyme to pass through the pylorus. Passage of chyme through the pylorus into the duodenum causes duodenal peristalsis, which causes the duodenal unit to pull the pyloric plug against the pylorus, and/or against the wall of the antrum surrounding the pylorus, as shown in FIG. 13C. This opened/closed cycling of the pylorus results in slow release of the chyme from the stomach into the duodenum.

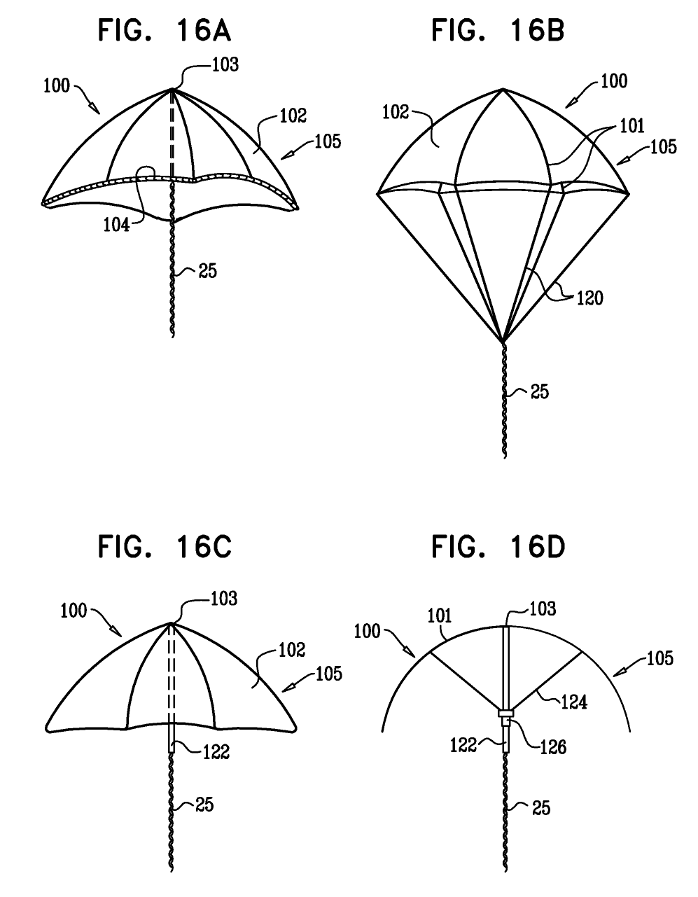

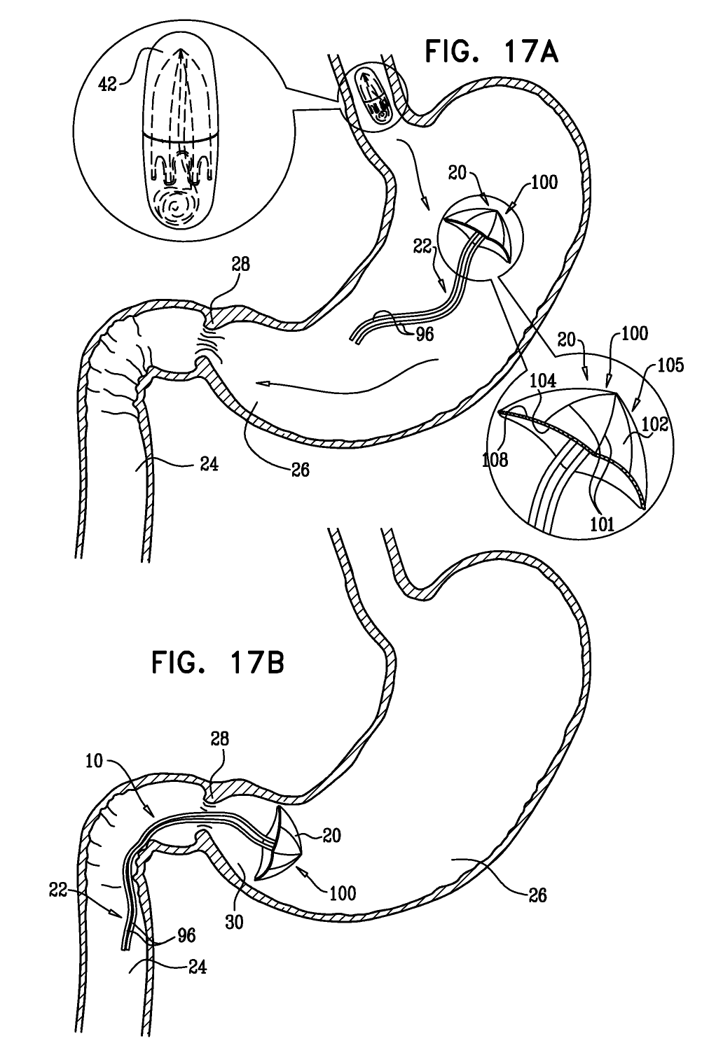

FIGS. 14A-B and 14C are schematic illustrations of yet another configuration of device 10 in an expanded state in the stomach and in an anchored position, respectively, in accordance with an application of the present invention. In this configuration, duodenal unit 22 comprises one or more elongated members 96, each of which typically has a length of at least 1 cm, or no more than 20 cm, or between 1 and 20 cm, e.g., 10 cm. For example, elongated members 96 may comprise strings, springs, tubes, ribbons, or a combination of such elements. For some applications, as shown in FIG. 14C, duodenal peristalsis pulls elongated members 96 distally in the duodenum, causing the members to pull the pyloric plug against the pylorus, and/or against the wall of the antrum surrounding the pylorus. Alternatively, elongated members 96 may be provided in applications described herein in which the gastric anchor is shaped so as to define passage 40 therethrough, such as described with reference to FIGS. 1-3, 11A-C, and/or 12. Although duodenal unit 22 is shown as comprising exactly two elongated members 96 in FIGS. 14A-C, for some applications the duodenal unit comprises exactly one elongated member 96, or three or more elongated members.

Although gastric anchor 20 is shown in FIGS. 14A-C shaped as a sphere, the gastric anchor may instead be configured with any of the other configurations shown or described herein (e.g., with reference to FIG. 9, 10A-B, 11A-C, 12, 13A-C, 15A-C, 16A-D, 17A-C, 18A-D, or 19A-B), or more generally, as any structure that assumes contracted and expanded states, including those described hereinabove with reference to FIGS. 13A-C. The anchor may have the dimensions described hereinabove with reference to FIGS. 13A-C.

For some applications, this configuration of treatment device 10 results in intermittent, alternating periods of peristalsis in the duodenum, and corresponding periods of full or partial blocking of the pylorus. During periods in which chyme is not in the duodenum, and thus peristalsis does not occur, elongated members 96 do not hold the pyloric plug against the pylorus. Natural muscular activity of the stomach moves the pyloric plug away from the pylorus, again assuming the position shown by way of example in FIG. 14B, and allowing chyme to pass through the pylorus. Passage of chyme through the pylorus into the duodenum causes duodenal peristalsis, which causes the elongated members to pull the pyloric plug against the pylorus, and/or against the wall of the antrum surrounding the pylorus, as shown in FIG. 14C. This opened/closed cycling of the pylorus results in slow release of the chyme from the stomach into the duodenum.

FIGS. 14D-F are schematic illustrations of additional configurations of elongated members 96, in accordance with respective applications of the present invention. In these configurations, elongated members 96 are configured to expand upon coming in contact with a duodenal liquid (which is acidic, but less acidic than gastric liquid). Such expansion may inhibit motion of chyme into and/or through the duodenum, which may slow gastric emptying and/or inhibit absorption of nutrients. These configurations may be used in applications described herein in which gastric anchor 20 is configured to serve as a pyloric plug, such as described with reference to FIGS. 9-10B, 14A-C, 16A-D, 17A-C, 18A-D, and/or 19A-B, or in applications described herein in which the gastric anchor is shaped so as to define passage 40 therethrough, such as described with reference to FIGS. 1-3, 11A-C, and/or 12. Although duodenal unit 22 is shown as comprising exactly two elongated members 96 in FIGS. 14D-F, for some applications the duodenal unit comprises exactly one elongated member 96, or three or more elongated members. Optionally, duodenal unit 22 comprises both elongated members 96 and at least one non-elongated element coupled to at least a portion of the elongated members 96, typically near a distal end of the elongated members. For example, the non-elongated element may be similar to the configuration of duodenal unit 22 shown in FIG. 3. For some applications, the non-elongated element has a volume of at least 0.2 cc, or a volume of no more than 10 cc, or a volume of between 0.2 and 10 cc.

For example, elongated members 96 may comprise a material (e.g., a gel, a sponge (such as a collagen sponge), e.g., containing a hydroscopic material, such as algae, or bicarbonate) that swells upon contact with the liquid contents of the duodenum, such as shown in FIGS. 14D and 14E, and/or a balloon or a sponge that fills with a gas upon contact with the liquid contents of the duodenum, such as shown in FIG. 14F. Optionally, elongated members 96 are interconnected, such as by thin elements, e.g., strings or sponges, which may further inhibit motion of chyme.

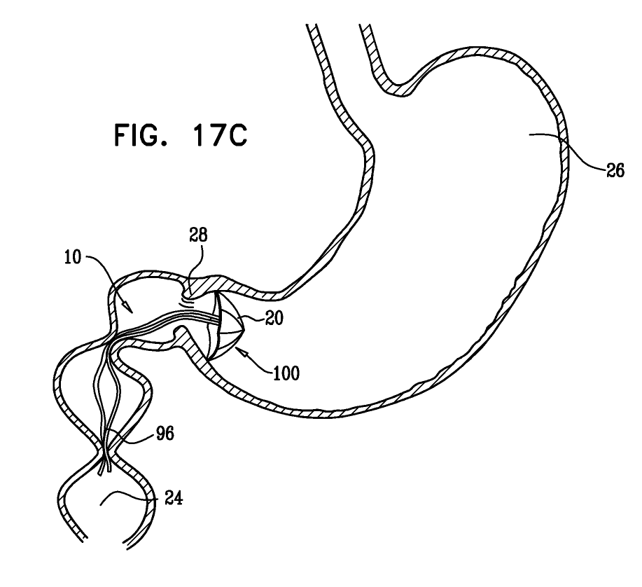

FIGS. 15A-B and 15C are schematic illustrations of still another configuration of device 10 in an expanded state in the stomach and in an anchored position, respectively, in accordance with an application of the present invention. In this configuration, gastric anchor 20 comprises a pyloric plug 100, which, when in an expanded state, is bowl-shaped, i.e., is concave with an opening on one side. For some applications, bowl-shaped pyloric plug 100 is generally umbrella-shaped and/or approximately hemispherical. Pyloric plug 100 typically comprises a flexible sheet 102, which, for example, may comprise silicone or plastic (e.g., polyurethane).

For some applications, pyloric plug 100 comprises a frame 150 to which flexible sheet 102 is coupled. Frame 105 comprises a plurality of ribs 101. Ribs 101 typically comprise wires, arranged to intersect with each other at a central point at an apex 103 of frame 105, such that each wire defines two of the ribs. For example, the frame may comprise between three and 36 wires, typically between 16 and 30, such that the frame comprises between six and 72 ribs, typically between 32 and 60. The wires may comprise a metal, such as Nitinol, or a plastic. The ribs are typically configured such that pyloric plug 100 assumes the expanded state when the ribs are in their resting state. Alternatively, pyloric plug 100 assumes and maintains its bowl shape due to accordion folding of flexible sheet 102, which is sufficiently rigid to provide accordion pleats.

The bowl-shaped structure provided by frame 150 generally prevents inversion of the plug and passage thereof through the pylorus by peristalsis.

As shown in the blow-up in FIG. 15A, the gastric anchor is swallowed in a contracted state, in which ribs 101 are in a compressed state, squeezed toward each other, such as by enclosure 42, if provided, or by one or more other dissolvable elements (configuration not shown). After being swallowed, entering stomach 26, and coming in contact with stomach contents, anchor 20 expands as ribs 101 assume their expanded state, as shown in FIG. 15A. In its expanded state, the anchor is prevented from passing through pylorus 28 even when the pylorus is in an open, relaxed state. More generally, pyloric plug 100 is configured to initially assume a contracted size, and, upon coming in contact with a liquid, to expand sufficiently to prevent passage of the anchor through a round opening having a diameter of between 1 cm and 3 cm. For some applications, when in its expanded state, pyloric plug 100 has a greatest cross-section of at least 2 cm, typically at least 3 cm, to prevent passage through the pylorus.

As shown in FIG. 15B, peristalsis advances treatment device 10 toward pylorus 28, and duodenal unit 22 into duodenum 24. As shown in FIG. 15C, duodenal peristalsis advances the duodenal unit distally in the duodenum. As a result of this advancement, the duodenal unit pulls the pyloric plug (such as via tether 25) toward the pylorus, such that the pyloric plug at least partially blocks the pylorus, such as entirely blocks the pylorus.

For some applications, this configuration of treatment device 10 results in intermittent, alternating periods of peristalsis in the duodenum, and corresponding periods of full or partial blocking of the pylorus. During periods in which chyme is not in the duodenum, and thus peristalsis does not occur, duodenal unit 22 does not hold the pyloric plug against the pylorus. Natural muscular activity of the stomach moves the pyloric plug away from the pylorus, again assuming the position shown by way of example in FIG. 15B, and allowing chyme to pass through the pylorus. Passage of chyme through the pylorus into the duodenum causes duodenal peristalsis, which causes the duodenal unit to pull the pyloric plug against the pylorus, and/or against the wall of the antrum surrounding the pylorus, as shown in FIG. 15C. This opened/closed cycling of the pylorus results in slow release of the chyme from the stomach into the duodenum.

Flexible sheet 102 typically has an area of at least 3 cm2, such that pyloric plug 100 at least partially blocks the pylorus. In addition, flexible sheet 102 typically has an area of less than 30 cm2, such that pyloric plug 100 does not apply any pressure to the wall of the stomach, except perhaps in a limited area of the antrum surrounding the pylorus. For some applications, the flexible sheet has an area of at least 3 cm2 and less than 30 cm2.

For some applications, pyloric plug 100 further comprises a band 104 that helps prevent possible inversion of the pyloric plug. For example, the band may be coupled to a rim 108 of bowl-shaped pyloric plug 100, around the perimeter thereof, and may partially or completely coincide with the perimeter. The band typically comprises a material that is stiffer than that of flexible sheet 102.

Typically, rim 108 of bowl-shaped pyloric plug 100 has a perimeter of at least 3 cm, no more than 12 cm, and/or between 3 cm and 12 cm. For some applications, rim 108 generally defines a plane (configuration not shown), while for other applications, portions of the rim between ribs 101 are slightly spaced from a plane defined by the ends of ribs 101, similar to the shape of a conventional umbrella-cloth (as shown in the figures).

For some applications, pyloric plug 100 is shaped so as to define one or more passages therethrough, in order to partially, rather than fully, block pylorus 28. Chyme is able to pass through the passages.

For some applications, pyloric plug 100 is configured to expand by comprising a material (e.g., a gel, a sponge, or bicarbonate) that swells upon contact with the liquid contents of the stomach, and/or a balloon or a sponge that fills with a gas upon contact with the liquid contents of the stomach. Alternatively, pyloric plug 100 uses other chemical and/or mechanical techniques for expansion.

Reference is made to FIGS. 16A-D, which are schematic illustrations of several configurations of pyloric plug 100, in accordance with respective applications of the present invention. In the configuration shown in FIG. 16A (also shown in FIGS. 15A-C), tether 25 is coupled to an inner surface of pyloric plug 100 near (i.e., within 2 mm of) of apex 103, such as at the apex.

In the configuration shown in FIG. 16B, tether 25 comprises a plurality of connecting sub-tethers 120, which couple the tether to a respective plurality of sites of pyloric plug 100. For example, sub-tethers 120 may be coupled to respective ones of ribs 101, either at the respective ends of the ribs (as shown), or elsewhere along the ribs (configuration not shown). For some applications, the number of sub-tethers equals the number of ribs, while for other applications, the number of sub-tethers is less than the number of ribs. For still other applications, the number of sub-tethers is greater than the number of ribs.

In the configuration shown in FIG. 16C, pyloric plug 100 comprises a post 122, a first end of which is coupled to an inner surface of pyloric plug 100 near (i.e., within 2 mm of) of apex 103, such as at the apex. The second end of the post is coupled to tether 25, thereby indirectly coupling the tether to the pyloric plug. The post may be rigid, semi-rigid, or flexible.

In the configuration shown in the cross-sectional illustration of FIG. 16D (in which, for clarity of illustration, flexible sheet 102 is not shown), pyloric plug 100 further comprises a plurality of stretchers 124, which provide the frame with additional structural strength. The stretchers are coupled to post 122 and respective ones of the ribs. Optionally, the pyloric plug further comprises a runner 126, which slides along post 122, as is known in the umbrella art. One end of each of the stretchers is coupled to runner 126, such that the stretchers are indirectly coupled to post 122.

FIGS. 17A-B and 17C are schematic illustrations of another configuration of device 10 in an expanded state in the stomach and in an anchored position, respectively, in accordance with an application of the present invention. In this configuration, anchor 20 comprises pyloric plug 100, such as described hereinabove with reference to FIGS. 15A-C and 16A-D, with the differences described hereinbelow. Duodenal unit 22 comprises one or more elongated members 96. Each of the elongated members typically has a length of at least 1 cm, or no more than 20 cm, or between 1 and 20 cm, e.g., 10 cm. For example, elongated members 96 may comprise strings, springs, tubes, ribbons, or a combination of such elements. For some applications, as shown in FIG. 17C, duodenal peristalsis pulls elongated members 96 distally in the duodenum, causing the members to pull the pyloric plug against the pylorus, and/or against the wall of the antrum surrounding the pylorus. Although duodenal unit 22 is shown as comprising exactly two elongated members 96 in FIGS. 17A-C, for some applications the duodenal unit comprises exactly one elongated member 96, or three or more elongated members.

For some applications, this configuration of treatment device 10 results in intermittent, alternating periods of peristalsis in the duodenum, and corresponding periods of full or partial blocking of the pylorus. During periods in which chyme is not in the duodenum, and thus peristalsis does not occur, elongated members 96 do not hold the pyloric plug against the pylorus. Natural muscular activity of the stomach moves the pyloric plug away from the pylorus, again assuming the position shown by way of example in FIG. 17B, and allowing chyme to pass through the pylorus. Passage of chyme through the pylorus into the duodenum causes duodenal peristalsis, which causes the elongated members to pull the pyloric plug against the pylorus, and/or against the wall of the antrum surrounding the pylorus, as shown in FIG. 17C. This opened/closed cycling of the pylorus results in slow release of the chyme from the stomach into the duodenum.

For some applications, elongated members 96 are coupled to pyloric plug 100 near (i.e., within 2 mm of) of apex 103, such as at the apex. Alternatively, the elongated members are coupled to other sites of the pyloric plug, such as described hereinabove with reference to FIGS. 16B, 16C, 16C, and/or 16D, mutatis mutandis. For some applications, elongated members 96 are coupled directly to pyloric plug 100, while for other applications, the elongated members are coupled indirectly to the pyloric plug, such as via tether 25, sub-tethers 120, and/or post 122.

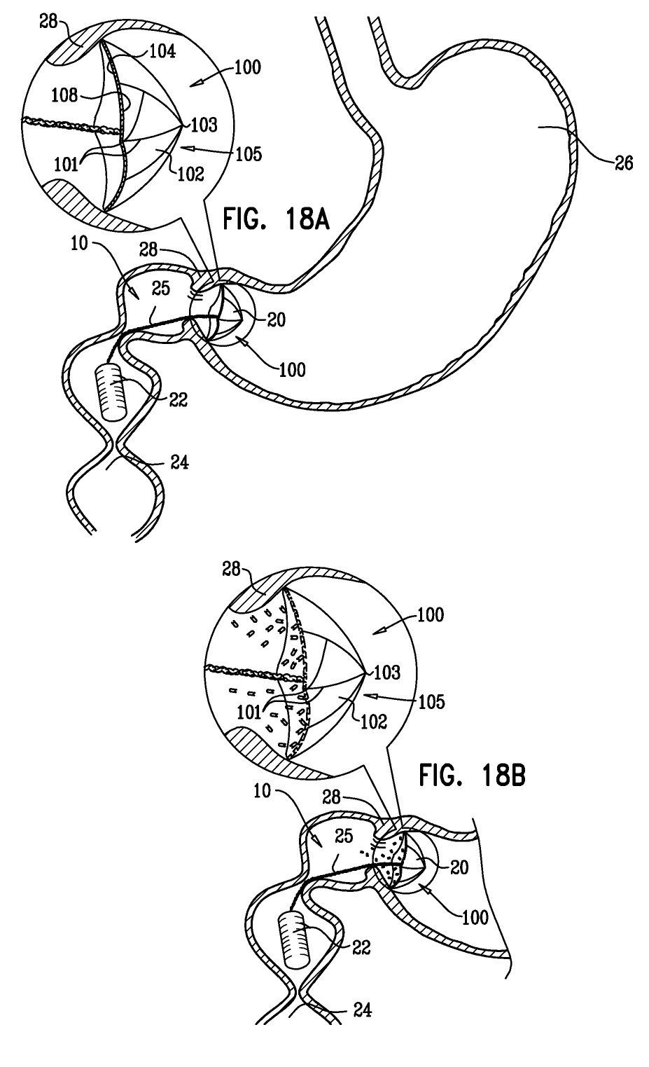

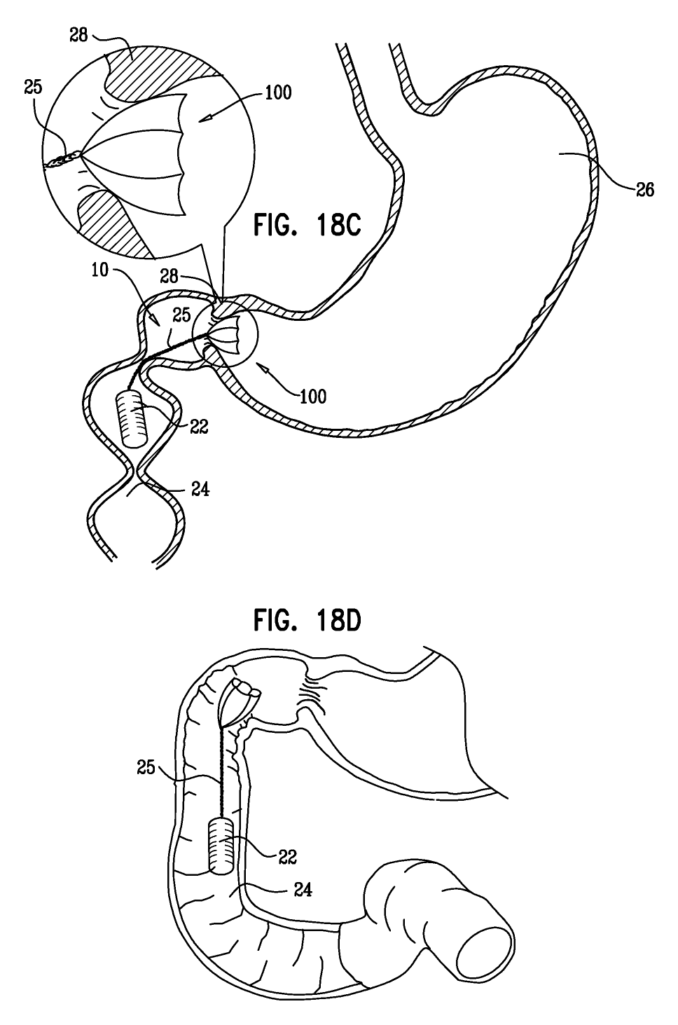

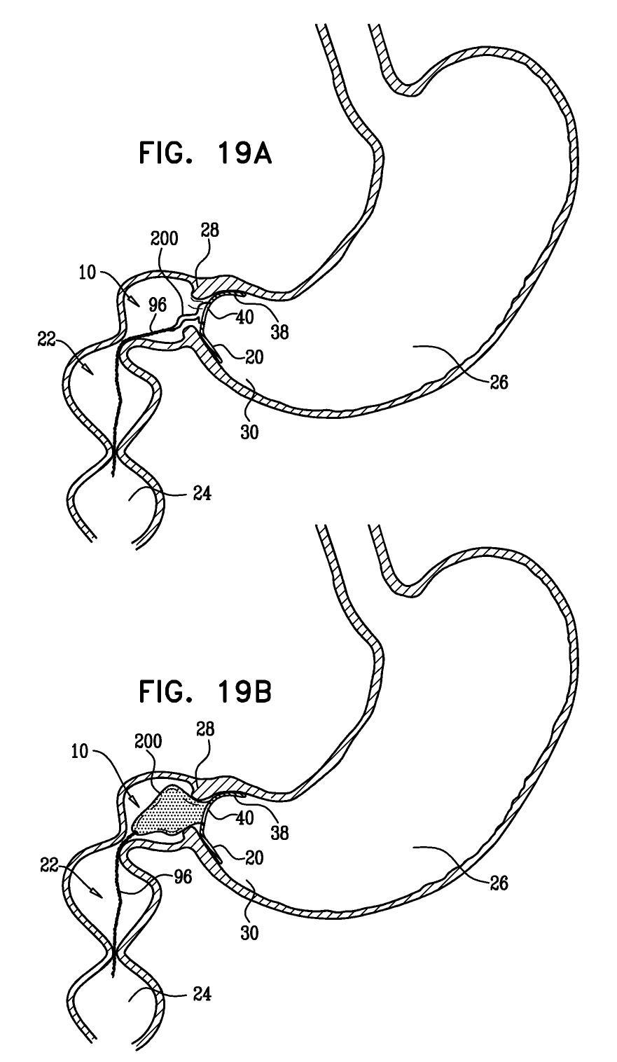

Reference is made to FIGS. 18A-D, which are schematic illustrations of a configuration of pyloric plug 100 anchored in the stomach and passing through the pylorus, in accordance with an application of the present invention. This configuration may be implemented in combination with the configurations of pyloric plug 100 described herein with reference to FIGS. 15A-C, 16A-D, 17A-C, and/or 19A-B. As mentioned above with reference to FIGS. 15A-C, for some applications pyloric plug 100 further comprises band 104 that helps prevent possible inversion of the pyloric plug. For example, the band may be coupled to rim 108 of bowl-shaped pyloric plug 100, around the perimeter thereof, and may partially or completely coincide with the perimeter. The band typically comprises a material that is stiffer than that of flexible sheet 102.

For some applications, band 104 is biodegradable, as shown in FIG. 18B. Upon degrading, typically a number of hours after the device has been swallowed, the band no longer prevents inversion of bowl-shaped pyloric plug 100, as shown in FIG. 18C. When inverted, the shape of pyloric plug 100 no longer prevents the plug from passing through pylorus 28. Peristalsis thus causes the plug to pass through the pylorus and be evacuated through the GI tract. Alternatively, bowl-shaped pyloric plug 100 inverts as it is pulled against and then into pylorus 28 by peristalsis after the band has degraded.

Alternatively or additionally, ribs 101, described hereinabove with reference to FIG. 15A, are biodegradable. When the ribs degrade, the device passes through the pylorus and GI tract. Still further alternatively or additionally, elements of pyloric plug 100 that couple together ribs 101 biodegrade.

Reference is now made to FIGS. 19A-B, which are schematic illustrations of another configuration of swallowable medical treatment device 10 in which the device serves as a pyloric plug, in accordance with an application of the present invention. FIG. 19A shows treatment device 10 in an anchored position, before expansion of a duodenal plug component 200. FIG. 19B shows the treatment device after expansion of the duodenal plug component. In this configuration, gastric anchor 20 is configured to serve as a pyloric plug, which is configured to at least partially block pylorus 28 upon being pulled toward the duodenum by duodenal unit 22. Gastric anchor 20 comprises duodenal plug component 200 that is at least partially inserted into the pylorus, i.e., at least partially passes through the pylorus into the duodenum. Typically, duodenal plug component 200 is configured to expand in the duodenum, and thus at least partially block the pylorus, optionally together with a portion of gastric anchor 20. Such blockage of the pylorus induces a sensation of satiety, e.g., by slowing the passage of chyme from the stomach. The device thus may slow a rise in blood sugar during and after eating food. The device is thus useful for treating conditions such as obesity and diabetes.

In this configuration, duodenal unit 22 is typically coupled to gastric anchor 20 via duodenal plug component 200. For some applications, duodenal unit 22 comprises one or more elongated members 96, which are coupled to duodenal plug component 200. Duodenal peristalsis pulls the elongated members, which in turn pull on the duodenal plug component, holding the duodenal plug component and the rest of gastric anchor 20 in place.

For expansion, duodenal plug component 200 may comprise a material (e.g., a gel, a sponge, or bicarbonate) that swells upon contact with the liquid contents of the duodenum, and/or a balloon or a sponge that fills with a gas upon contact with the liquid contents of the duodenum (which is acidic, but less acidic than gastric liquid). Alternatively, duodenal plug component 200 uses other chemical and/or mechanical techniques for expansion.

For some applications, gastric anchor 20 is not shaped so as to define a passage therethrough, and thus may help duodenal plug component 200 to block the pylorus. For example, gastric anchor 20 may implement techniques described hereinabove with reference to FIGS. 10A-B, 13A-C, 14A-C, 14D-F, 15A-C, 16A-D, 17A-C, and 18A-D.

For other applications, gastric anchor 20 is shaped so as to define passage 40 therethrough, such as a hole (e.g., a central hole), such as described hereinabove with reference to FIGS. 1-3, 11A-C, and 12. In this configuration, stomach contents, such as chyme and liquid, may pass through the opening into duodenal plug component 200, thereby expanding the component.

Although duodenal unit 22 is shown as comprising exactly one elongated member 96 in FIGS. 19A-B, for some applications the duodenal unit comprises two or more elongated members 96.

Optionally, duodenal unit 22 further comprises at least one non-elongated element coupled to at least a portion of the elongated members 96, typically near a distal end of the elongated members. For example, the non-elongated element may be similar to the configuration of duodenal unit 22 shown in FIG. 3. For some applications, the non-elongated element has a volume of at least 0.2 cc, or a volume of no more than 10 cc, or a volume of between 0.2 and 10 cc.

For some applications, elongated members 96 are configured to expand, such as using techniques described hereinabove with reference to FIGS. 14D-F.

For some applications, a variety of treatment devices are provided, calibrated based on time of disintegration and/or size of the passage through the pyloric plug (for applications in which the passage is provided). The physician selects the most appropriate calibration, based on the individual patient's condition and/or pyloric orifice size.

For some applications, such as those described hereinabove with reference to FIG. 1-3, 9, 10A-B, 11A-C, 12, 13A-C, 15A-C, 18A-D, or 19A-B, duodenal unit 22 is configured to expand upon coming in contact with a liquid, such as stomach and/or duodenal contents. Such expansion increases the surface area of the duodenal unit that comes in contact with the wall of the duodenum, thereby increasing the pulling force of duodenal peristalsis on the duodenal unit. For example, the duodenal unit may comprise a material (e.g., a gel, a sponge, or bicarbonate) that swells upon contact with liquid, and/or a balloon or a sponge that fills with a gas upon contact with liquid.

For some applications, such as those described hereinabove with reference to FIG. 1-3, 7, 8, 9, 10A-B, 11A-C, 12, 13A-C, 14A-C, 14D-F, 15A-C, 17A-C, 18A-D, or 19A-B, the anchor is configured to expand by comprising a material (e.g., a gel, a sponge, or bicarbonate) that swells upon contact with the liquid contents of the stomach, and/or a balloon or a sponge that fills with a gas upon contact with the liquid contents of the stomach. Alternatively, gastric anchor 20 uses other chemical and/or mechanical techniques for expansion.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising a swallowable medical treatment device, which is configured to initially assume a contracted state having a volume of less than 4 cm3, and which comprises:
a gastric anchor, which initially assumes a contracted size, and which is configured to, upon coming in contact with a liquid, expand sufficiently to prevent passage of the gastric anchor through a round opening having a diameter of between 1 cm and 3 cm; and
a duodenal unit, which is configured to pass through the opening, and which is coupled to the gastric anchor such that the duodenal unit is held between 1 cm and 20 cm from the gastric anchor.

2. (canceled)

3. The apparatus according to claim 1, further comprising a tether, which couples the duodenal unit to the gastric anchor, and has a length of between 1 cm and 20 cm.

4. The apparatus according to claim 1, wherein the duodenal unit comprises one or more elongated members, each of which has a length of between 1 and 20 cm.

5. The apparatus according to claim 4, wherein the elongated members are configured to expand upon coming in contact with a duodenal liquid.

6. The apparatus according to claim 5, wherein the elongated members comprise sponges.

7. The apparatus according to claim 5, wherein the elongated members comprise balloons.

8-11. (canceled)

12. The apparatus according to claim 1, wherein the gastric anchor comprises a flexible sheet which initially is rolled around at least a portion of the duodenal unit to assume the contracted size, and which is configured to prevent the passage of the anchor through the opening by unrolling upon coming in contact with the liquid.

13-14. (canceled)

15. The apparatus according to claim 1, wherein the gastric anchor is configured to serve as a plug, which is configured to at least partially block the opening.

16. The apparatus according to claim 15, wherein the gastric anchor comprises a duodenal plug component, which is configured to at least partially pass through the opening, and expand upon coming in contact with a duodenal liquid, so as to at least partially block the opening.

17. The apparatus according to claim 16, wherein the duodenal unit is coupled to the gastric anchor via the duodenal plug component.

18. The apparatus according to claim 17, wherein the duodenal unit comprises one or more elongated members, which are coupled to the duodenal plug component.

19. The apparatus according to claim 15, wherein the duodenal unit comprises one or more elongated members, each of which has a length of between 1 and 20 cm, and which are configured to expand upon coming in contact with a duodenal liquid.

20. The apparatus according to claim 15, wherein the gastric anchor comprises a flexible sheet.

21-29. (canceled)

30. The apparatus according to claim 15, wherein the device is configured to intermittently at least partially block the opening.

31-35. (canceled)

36. The apparatus according to claim 1, further comprising a dissolvable enclosure that entirely surrounds the swallowable medical treatment device when the device initially assumes the contracted state.

37. The apparatus according to claim 1,
wherein the opening is a pylorus of a subject,
wherein the liquid is stomach contents of the subject,
wherein the gastric anchor is configured to, upon coming in contact with the stomach contents, expand sufficiently to prevent passage of the anchor through the pylorus, and
wherein the duodenal unit is configured to pass through the pylorus, and is coupled to the gastric anchor such that the duodenal unit is held in a duodenum of the subject.

38. The apparatus according to claim 37, wherein the gastric anchor is configured to at least partially biodegrade in a stomach of a subject, so as to allow passage of the anchor through the pylorus after a period of time.

39. Apparatus comprising a swallowable medical treatment device, which is configured to initially assume a swallowable contracted state, and which comprises:
a gastric anchor, which initially assumes a contracted size, and which is configured to, upon coming in contact with stomach contents of a subject, expand sufficiently to prevent passage of the anchor through a pylorus of the subject even when the pylorus is in an open, relaxed state; and
a duodenal unit, which is configured to pass through the pylorus into a duodenum of the subject, and which is coupled to the gastric anchor such that the duodenal unit is held in the duodenum.

40. The apparatus according to claim 39, further comprising a tether, which couples the duodenal unit to the gastric anchor, and has a length of between 1 cm and 20 cm.

41. The apparatus according to claim 39, wherein the duodenal unit comprises one or more elongated members, each of which has a length of between 1 and 20 cm.

42. The apparatus according to claim 41, wherein the elongated members are configured to expand upon coming in contact with a duodenal liquid.

43. The apparatus according to claim 39, wherein the gastric anchor is configured to serve as a pyloric plug, which is configured to at least partially block the pylorus.

44. The apparatus according to claim 43, wherein the gastric anchor comprises a duodenal plug component, which is configured to at least partially pass through the pylorus, and expand upon coming in contact with a duodenal liquid, so as to at least partially block the pylorus.

45. The apparatus according to claim 44, wherein the duodenal unit is coupled to the gastric anchor via the duodenal plug component.

46. The apparatus according to claim 45, wherein the duodenal unit comprises one or more elongated members, which are coupled to the duodenal plug component.

47. The apparatus according to claim 43, wherein the gastric anchor comprises a flexible sheet.

48-54. (canceled)

55. The apparatus according to claim 39, further comprising a dissolvable enclosure that entirely surrounds the swallowable medical treatment device when the device initially assumes the swallowable contracted state.

56-65. (canceled)

66. A method comprising:
receiving, by a subject, a swallowable medical treatment device in an initially contracted state, which device includes a gastric anchor having an initially contracted size, and a duodenal unit coupled to the gastric anchor; and
swallowing the treatment device by the subject, so that the anchor, upon coming in contact with stomach contents of the subject, expands sufficiently to prevent passage of the anchor through a pylorus of the subject, and the duodenal unit passes through the pylorus into a duodenum of the subject and is held in the duodenum by the anchor.

67. The method according to claim 66, wherein receiving comprises receiving the swallowable treatment device that further includes a tether, which couples the duodenal unit to the gastric anchor, and has a length of between 1 cm and 20 cm.

68-70. (canceled)

71. The method according to claim 66, wherein receiving comprises receiving the swallowable treatment device in which the gastric anchor comprises a flexible sheet which initially is rolled around at least a portion of the duodenal unit to assume the initially contracted size, and which is configured to prevent the passage of the anchor through the pylorus by unrolling upon coming in contact with the stomach contents.

72-73. (canceled)

74. The method according to claim 66, wherein the gastric anchor is configured to serve as a pyloric plug, and wherein swallowing comprises swallowing the treatment device such that the pyloric plug at least partially blocks the pylorus.

75. The method according to claim 74, wherein the gastric anchor includes a duodenal plug component, which is configured to at least partially pass through the pylorus, and wherein swallowing comprises swallowing the treatment device such that the duodenal plug component at least partially passes through the pylorus, and expands upon coming in contact with a duodenal liquid, so as to at least partially block the pylorus.

76. The method according to claim 75, wherein the duodenal unit is coupled to the gastric anchor via the duodenal plug component.

77. The method according to claim 76, wherein the duodenal unit includes one or more elongated members, which are coupled to the duodenal plug component.

78. The method according to claim 74, wherein receiving comprises receiving the treatment device in which the plug comprises a flexible sheet.

79-81. (canceled)

82. The method according to claim 74, wherein swallowing comprises swallowing the treatment device such that the pyloric plug intermittently at least partially blocks the pylorus.

83-89. (canceled)
